SEQUENCE LISTING (8 sequences)

SEQ ID NO: 1 - protein, 42 aa, Homo sapiens [sequence omitted]

SEQ ID NO: 2 - protein, 43 aa, Homo sapiens
DAEFRHDSGYEVHHQKLVFFAEDVGSNKGAIIGLMVGGVVIAT

SEQ ID NO: 3 - protein, 40 aa, Homo sapiens
DAEFRHDSGYEVHHQKLVFFAEDVGSNKGAIIGLMVGGVV

SEQ ID NO: 4 - protein, 83 aa, Homo sapiens
LVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - protein, 99 aa, Homo sapiens
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 6 - protein, 758 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQEPESGKVVQEGFLREPGPPGLSHQLMSGMPGAPLLPEGPREATRQPSGTGPEDTEGGRHAPELLKHQLLGDLHQEGPPLKGAGGKERPGSKEEVDEDRDVDESSPQDSPPSKASPAQDGRPPQTAAREATSIPGFPAEGAIPLPVDFLSKVSTEIPASEPDGPSVGRAKGQDAPLEFTFHVEITPNVQKEQAHSEEHLGRAAFPGAPGEGPEARGPSLGEDTKEADLPEPSEKQPAAAPRGKPVSRVPQLKARMVSKSKDGTGSDDKKAKTSTRSSAKTLKNRPCLSPKHPTPGSSDPLIQPSSPAVCPEPPSSPKYVSSVTSRTGSSGAKEMKLKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 8 - protein, 140 aa, Homo sapiens
MDVFMKGLSKAKEGVVAAAEKTKQGVAEAAGKTKEGVLYVGSKTKEGVVHGVTTVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2016/0136229 A1
(43) Pub. Date: May 19, 2016

(54) MATERIALS FOR POSITIVE CATHEPSIN B MODULATION AND METHODS OF USE FOR TREATING MILD COGNITIVE IMPAIRMENT (MCI), EARLY DEMENTIA, A-SYNUCLEINOPATHY, TRAUMATIC BRAIN INJURY, CARDIOMYOPATHY, EYE DISEASE AND SKIN DAMAGE

(71) Applicants: Ben A. Bahr, Lake Waccamaw, NC (US); Uzoma S. Ikonne, Mesa, AZ (US)

(72) Inventors: Ben A. Bahr, Lake Waccamaw, NC (US); Uzoma S. Ikonne, Mesa, AZ (US)

(73) Assignee: University of North Carolina at Pembroke, Pembroke, NC (US)

(21) Appl. No.: 14/899,391

(22) PCT Filed: Jun. 17, 2014
(86) PCT No.: PCT/US2014/042728

§ 371 Date: Dec. 17, 2015

Related U.S. Application Data
(60) Provisional application No. 61/836,216, filed on Jun. 18, 2013.

(51) Int. Cl. A61K 38/05; A61K 38/08; A61K 38/06; A61K 31/365; A61K 31/27; A61K 31/655

(52) U.S. Cl. CPC A61K 38/05; A61K 31/27; A61K 31/655; A61K 38/06; A61K 31/365; A61K 38/08

(57) ABSTRACT

The present invention provides methods for treating a subject afflicted with i) Mild Cognitive Impairment (MCI), ii) early dementia, iii) early α-synucleinopathy, iv) traumatic brain injury, v) cardiomyopathy, vi) eye disease, or vii) skin damage, comprising administering to the subject at least one compound that increases the level of active cathepsin B in cells of the subject, or a pharmaceutically acceptable salt or ester thereof, in an amount that is effective to treat the subject. The present invention also provides compositions for treating a subject afflicted with i) Mild Cognitive Impairment (MCI), ii) early dementia, iii) early α-synucleinopathy, iv) traumatic brain injury, v) cardiomyopathy, vi) eye disease, or vii) skin damage.

This application claims priority of U.S. Provisional Patent Application No. 61/836,216, filed Jun. 18, 2013, the entire content of which is hereby incorporated herein by reference.

This application incorporates-by-reference nucleotide and/or amino acid sequences which are present in the file named “140617_7335_85280-A-PCT_SEQUENCELISTING_REB.TXT”, which is 14.7 kilobytes in size, and which was created Jun. 17, 2014 in the IBM-PC machine format, having an operating system compatibility with MS-Windows, which is contained in the text file filed Jun. 17, 2014 as part of this application.

Throughout this application, various publications are referenced, including referenced in parenthesis. Full citations for publications referenced in parenthesis may be found listed at the end of the specification immediately preceding the claims. The disclosures of all referenced publications in their entireties are hereby incorporated by reference into this application in order to more fully describe the state of the art to which this invention pertains.

### BACKGROUND OF INVENTION

Lysosomes are the cellular components involved in removing misfolded/aggregating proteins, but with aging lysosomes become less effective at clearing toxic accumulations that are linked to the deterioration of neuronal connections. Protein accumulation disorders, including Alzheimer's disease (AD), frontotemporal dementia (FTD), Parkinson's disease (PD), Huntington's disease (HD), amyotrophic lateral sclerosis (ALS), and other dementias, are suspected to involve imbalances between protein production and protein clearance. For example, synaptic pathology that disrupts neuronal connectivity has long been considered the key event in age-related disorders that 1) leads to cognitive deficits and 2) contributes to early, gradual changes that constitute risk factors for dementia.

Heart failure is the leading cause of death in the developed world, and it represents a common endpoint for several diseases, including hypertension, coronary artery disease, and the cardiomyopathies. A lack of pathogenic commonality is underscored by the large number of mutations in different classes of cardiac proteins that have been linked to dilated and hypertrophic cardiomyopathy (HCM). Amyloid oligomers are present in cardiomyocytes derived from human heart-failure subjects and in animal models of cardiomyopathy. However, the increased expression of some lysosomal proteins may contribute to heart failure. Myocardial expression of cathepsin B has been found in subjects with heart failure (Ge et al. (2006) “Enhanced myocardial cathepsin B expression in subjects with dilated cardiomyopathy” Eur J Heart Fail. 8(3):284-9).

Improved methods for treating protein accumulation diseases are needed.

### SUMMARY OF THE INVENTION

The present invention provides new strategies for increasing cellular levels of active cathepsin B in order to enhance lysosomal activity, synaptic recovery, and cellular integrity in several tissue types. The present invention provides effective treatments for mild cognitive impairment (MCI), early dementia, α-synucleinopathies, traumatic brain injury, cardiomyopathies, eye disease, and skin damage.

The present invention provides methods for treating a subject afflicted with i) Mild Cognitive Impairment (MCI), ii) early dementia, iii) early α-synucleinopathy, iv) traumatic brain injury, v) cardiomyopathy, vi) eye disease, or vii) skin damage, comprising administering to the subject at least one compound that increases the level of active cathepsin B in cells of the subject, or a pharmaceutically acceptable salt or ester thereof, in an amount that is effective to treat the subject. A non-limiting example of such a cathepsin B modulatory compound is N-carbobenzyloxy-L-phenylalanyl-L-alanyl-diazomethylketone, herein abbreviated PADK.

Synonyms and other abbreviations for this compound include:

carbobenzoxycarbonyl-L-phenylalanyl-L-alanyl-diazomethylketone;
N-Cbz-L-phenylalanyl-L-alanyl-diazomethylketone;

### N-Cbz-L-Phe-L-Ala-diazomethylketone;

### N-Cbz-Phe-Ala-diazomethylketone;

Z-L-phenylalanyl-L-alanyl-diazomethylketone;

### Z-L-Phe-L-Ala-diazomethylketone;

### Z-Phe-Ala-diazomethylketone;

### Z-Phe-Ala-CHN₂;

### Z-FA-CHN₂;

### Z-Phe-Ala-DMK;

### Z-FA-DMK;

### ZPADK;

carbobenzoxycarbonyl-L-phenylalanyl-L-alanine-D-diazomethane;
N-Cbz-L-phenylalanyl-L-alanyl-diazomethane;

### N-Cbz-L-Phe-L-Ala-diazomethane;

### N-Cbz-Phe-Ala-diazomethane;

Z-L-phenylalanyl-L-alanyl-diazomethane;

### Z-L-Phe-L-Ala-diazomethane;

### Z-Phe-Ala-diazomethane;

benzyloxycarbonylphenylalanylalanine diazomethyl ketone;
N-benzyloxycarbonylphenylalanylalanine diazomethyl ketone;
N-benzyloxycarbonyl-L-phenylalanyl-L-alanyl diazomethyl ketone;
N-benzyloxycarbonyl-L-Phe-L-Ala diazomethyl ketone;
N-benzyloxycarbonyl-Phe-Ala diazomethyl ketone.
Other possible names for the PADK compound include:
(Z,3S)-1-diazonio-3-[[(2S)-3-phenyl-2-(phenylmethoxycarbonylamino)propanoyl]amino]but-1-en-2-olate
Carbamic acid, ((1S)-2-(((1S)-3-diazo-1-methyl-2-oxopropyl)amino)-2-oxo-1-(phenylmethyl)ethyl)-, phenylmethyl ester, (S—(R*,R*))—
Carbamic acid, (2-((3-diazo-1-methyl-2-oxopropyl)amino)-2-oxo-1-(phenylmethyl)ethyl)-, phenylmethyl ester, (S—(R*,R*))—

### CHEMBL2179950

### LS-186739

### LS-187426

71732-53-1

### AC1NUQLU

### CAS Registry Number (71732-53-1).

The present invention provides a method for treating a subject afflicted with i) Mild Cognitive Impairment (MCI) or ii) early dementia, comprising administering to the subject at least one compound that increases the level of active cathepsin B in brain cells of the subject, or a pharmaceutically acceptable salt or ester thereof, in an amount that is effective to treat the subject.

The present invention provides a method for treating a subject afflicted with cardiomyopathy comprising administering to the subject at least one compound that increases the level of active cathepsin B in heart cells of the subject, or a pharmaceutically acceptable salt or ester thereof, in an amount that is effective to treat the subject.

The present invention provides a method for treating a subject afflicted with i) a retinal disease, ii) an optic nerve disease, iii) retinal damage, or iv) optic nerve damage, comprising administering to the subject at least one compound that increases the level of active cathepsin B in eye cells of the subject, or a pharmaceutically acceptable salt or ester thereof, in an amount that is effective to treat the subject.

The present invention provides a method for treating a subject afflicted with traumatic brain injury (TBI), comprising administering to the subject at least one compound that increases the level of active cathepsin B in brain cells of the subject, or a pharmaceutically acceptable salt or ester thereof, in an amount that is effective to treat the subject.

The present invention provides a method for treating a subject afflicted with skin damage, comprising administering to the subject at least one compound that increases the level of active cathepsin B in skin or hypodermis cells of the subject, or a pharmaceutically acceptable salt or ester thereof, in an amount that is effective to treat the subject.

The present invention provides a method for treating a subject afflicted with a protein accumulation disease or TBI, comprising orally administering to the subject at least one compound that increases the level of active cathepsin B in cells of the subject, or a pharmaceutically acceptable salt or ester thereof, in an amount that is effective to treat the subject.

### BRIEF DESCRIPTION OF THE DRAWINGS

FIG. 1. Lysosomal modulation increases the cathepsin B content of lysosomes and reduces intracellular Δβ₁₋₄₂ in the APPswe/PS1ΔE9 transgenic mouse model of Alzheimer's disease. The lysosomal modulator Z-L-phenylalanyl-L-alanyl-diazomethylketone (PADK) has been shown to increase expression of procathepsin isoforms, as well as produce molecular changes consistent with enhanced cathepsin maturation and trafficking. Without wishing to be bound by any scientific theory, the latter steps may involve processing/trafficking of active intermediate (intCB) and mature forms (matCB) from early/late endosomes to lysosomes in order to promote protein degradation (A). To confirm localization, hippocampal tissue from PADK-treated mice (i.p., 20 mg/kg/day×9 days) that exhibit increased mature cathepsin B levels was stained with anti-cathepsin B (red) and anti-LAMP1 (green). The merged image shows that the PADK-modulated cathepsin B highly co-localizes with LAMP1-positive lysosomes in neurons (B). The increase in organellar cathepsin B enzyme (red) in PADK-treated APPswe/PS1ΔE9 transgenic mice (tg) was associated with a decrease in intracellular Δβ₁₋₄₂ in the CA1 pyramidal neurons (C). Arrows denote co-localization in lysosomes, capturing intact Δβ₁₋₄₂ before being degraded by cathepsin B. View-field widths: 12 μm. wt, vehicle-treated wild-type mice.

FIG. 2. The cathepsin B modulatory compound PADK improved learning and memory in 11-month Fischer rats, a model of mild cognitive impairment (MCI) and early dementia. A classic passive avoidance task to assess memory utilized a bi-sectioned apparatus consisting of a brightly-lit compartment and a dark compartment separated by a small door. In studies in the Bahr Lab, the 11-month rats exhibited a deficit in shock-induced passive avoidance learning as compared to young rats (P=0.012). In this figure, the female rats were injected daily with vehicle or 27 mg/kg PADK. Following each morning's injection, the rats were placed individually into the bright compartment and, after 10 s, the door was opened through which the rats typically entered immediately (3 min total time in apparatus/rat/day). Panel A: On the seventh injection day, latency time before entering the opened doorway was measured (pre-shock bars, mean±SEM), after which the door was closed to contain the animal in the dark compartment. A mild foot shock of 0.6 mA, 2 s duration, was then administered to the contained rat via the floor grid. Following the next day's injections, rats were again placed in the bright compartment and latency times measured after the door was opened (post-shock bars). The study shows that PADK-treated rats exhibit improved passive avoidance behavior as compared to aged rats that received vehicle only (**P=0.016). Panel B: The animal groups were also tested across consecutive days in the same novel field, and the number of center grid crossings per 3 min period was measured (mean±SEM), No significant difference was seen on day 1 of testing, however PADK animals exhibited improved exploratory habituation on the second day compared to vehicle-treated rats (*P<0.05). Brains were removed after the behavior studies in order to assess cathepsin B levels.

FIG. 3. Lysosomal modulator PADK dramatically up-regulates active cathepsin B levels in middle-aged Fischer rats, a model of MCI. The female rats were injected daily with vehicle (veh) or 27 mg/kg PADK for 9 days. Brain regions were dissected, homogenates prepared, and equal protein aliquots were assessed by immunoblot to label the 30-kDa active form of cathepsin B as well as actin load controls (A). The hippocampal homogenate samples were also stained for the postsynaptic marker GluR1 to assess for synaptic recovery in correspondence to cathepsin B regulation (A, lower blots). Active cathepsin B (B) and GluR1 levels (C) were plotted (mean±SEM), and both were found to be increased by PADK (***p<0.001; *p=0.0386).

FIG. 4. Enhancing lysosome efficiency with the cathepsin B modulator PADK leads to reductions of multiple types of proteinopathy-related accumulation events. (A) Vehicle-treated and PADK-treated mice (18 mg/kg/d×9 days, ip)(left) and (B) human neuronal cultures treated with control compound Z-FA or 10 μM PADK for 3 days (right) exhibited increased levels of active cathepsin B (intracellular green stain); view-field width: 100 and 300 μm, respectively. Age-matched wild-type mice (wt) and 9-10-month mutant human APP mice, a model of early dementia, treated with or without PADK were assessed for the 4-kDa Δβ peptide, the active form of cathepsin B (act CB), and other proteins in hippocampal samples (C). Similar treatment groups used another APP mouse model at 20-22 months of age to measure clearance of APP carboxyterminal fragments APP-αCTF and APP-βCTF (D). Rat hippocampal slice cultures from the untreated control group (con) and slices treated with submicromolar Δβ42 (early tauopathy model) in the absence or presence of PADK (5 μM for 4 days) were assessed for phosphorylated tau (pTau) and other proteins (E). For a model of early α-synucleinopathy, human A53T α-synuclein transgenic mice, at 7 months old, were studied with or without PADK treatment and assessed for human α-synuclein (α-syn) in TX-100 extracts of hippocampus and for other proteins in hippocampal homogenates (F). Plotted data (±SEM) were compared to neighboring vehicle groups by unpaired t-tests: *p=0.02, **p<0.01, ***p<0.001. APPswind and APP-PS1 mice are described in Butler et al., (2011), the entire content of which is hereby incorporated herein by reference.

FIG. 5. The cathepsin B modulator PADK also has a behavioral effect during early α-synucleinopathy. Vehicle-treated and PADK in the model of early α-synucleinopathy, human A53T α-synuclein transgenic mice, 7-8 months old, were injected with vehicle or PADK (18 mg/kg/day) for 11 days and their locomotor activity was measured in the open field test (OFT) for two days, center and perimeter crossings were measured. No difference in center crossings was found on day 1 as compared to wild-type mice (left graph), thus an indication of pre-symptomatic early stage Parkinson's disease (PD) before typical locomotor deficits. However, center crossings are often used as a measure of altered anxiety in PD mice. On day 2, mean center crossings±SEM are shown to be significantly increased in the A53T mice treated with vehicle (center graph; *p<0.05), indicating reduced anxiety at the early stage of PD pathology in the mouse model. PADK treated A53T mice did not consistently exhibit such a positive trend in center crossings on day 2, thus indicating that PADK offsets this component of PD pathology. The altered anxiety in the vehicle-treated A53T mice appears to be a selective effect since perimeter crossings on day 2 were unchanged (right graph).

FIG. 6. Cathepsin B modulation by PADK correlates with improved α-synuclein clearance across CNS regions. A53T mice expressing a mutant form of human α-synuclein were used at 7 months of age as a model of asymptomatic early α-synucleinopathy. The mouse model was injected daily with vehicle or the cathepsin B enhancing compound PADK (i.p., 18 mg/kg/day). Panel A: Wildtype (WT) and tg tissue extracts of different regions were tested by immunoblot, indicating a robust PADK-mediated increase in the active form of cathepsin B (act CB), and this was associated with reduced levels of human α-synuclein (α-syn). Active cathepsin B (8) and human α-synuclein levels (C) were plotted (mean±SEM), and PADK caused an increase in the former (*p<0.05 compared to WT, *p<0.02 to vehicle-treated tg mice) and a corresponding decrease in the latter (**p=0.01 compared to vehicle-treated A53T mice).

FIG. 7. The cathepsin B modulator PADK promotes synaptic integrity in the model of early α-synucleinopathy. The A53T transgenic and wildtype (wt) mice were injected daily with PADK or vehicle for 11 days. The 8-month A53T mice exhibited early α-synucleinopathy, linking α-synuclein accumulation with declines in synaptic proteins before indications of behavioral deficits (no differences were found between WT and A53T mice on tests of open field mobility, perimeter exploration, or center grid crossings). Staining the brain samples for other proteins found that the postsynaptic protein exhibited recovery in association with α-synuclein clearance (Figure C), and additional synaptic markers provided further evidence for recovery of synaptic integrity. Equal protein aliquots of hippocampal homogenates were analyzed by immunoblot for synaptic markers, showing PADK-improved levels of GluR1, GluR4, synaptophysin, and synapsin II; the actin load control was unchanged across the three groups (A). Mean GluR1 immunoreactivities±SEM are shown (B); post hoc test compared to vehicle-treated transgenics: *p<0.05. In addition, linear regression analysis found that, among the individual mouse samples, the percent recovery of the synaptic marker correlates significantly with the percent reduction in human α-synuclein (C).

FIG. 8. PADK also enhances cathepsin B species in the heart. The A53T early α-synucleinopathy mouse model was injected daily with vehicle or PADK (i.p., 18 mg/kg/day) to test for changes in heart tissue. (A) Wildtype (WT) and A53T transgenic (tg) heart tissue samples were tested by immunoblot, indicating a robust PADK-mediated increase in the active form of cathepsin B (CB-30, i.e. 30 kDa). The tg mice with protein accumulation stress exhibited dramatic declines in large anti-cathepsin B immunoreactive bands (CB-80, CB-85, CB-105), and the PADK treatment returned the high molecular weight species to near normal levels as found in WT mice (top). Positions of molecular weight standards (kDa) are shown on the left side of the immunoblot. (B) In the lower graph, ratios between CB-30 and CB-105 (mean±SEM) provide a good example of the unique PADK effect on cathepsin B species in the heart. **P<0.01 as compared to vehicle-treated A53T mice.

FIG. 9. The active form of cathepsin B is reduced in the slice model of Δβ-induced cardiomyopathy. Organotypic heart slice cultures from rats were treated with the control vehicle solution or with Δβ42, the peptide that accumulates in Alzheimer's disease brains as well as in heart tissue during some forms of cardiomyopathy. After 2 days of treatment, heart slices were harvested and equal protein aliquots tested for immunoreactivity levels (mean±SEM), indicating a significant decrease in active cathepsin B (*P=0.027). Such a detrimental effect on the lysosomal system suggests that a cathepsin B modulator like PADK would be beneficial to restore protein clearing capability during cardiomyopathy.

FIG. 10. The modulatory compound PADK up-regulates active cathepsin B in brain, eyes, and skin after oral dosing in rats. The rats were fed prepared portions of a peanut butter mixture containing an inactive peptide (ZFA) or PADK (providing a dose of 15 mg/kg), and this was done twice a day. No evidence of adverse effects was observed. Evidence of PADK crossing the BBB to enhance active cathepsin B levels in brain regions was found after 10 days of 15 mg PADK/kg/0.5 day. Four different brain regions were dissected and homogenates prepared, and equal protein aliquots were assessed by immunoblot to label the 30-kDa active form of cathepsin B (FC=frontal cortex). Eye and skin tissue preparations were also found to have significant increases in active cathepsin B after the PADK treatment.

FIG. 11. Safety evaluation of PADK found no evidence of toxicity. PADK did not cause bacterial cytotoxicity when tested at up to a 1000 times the IC₅₀ of the toxin mitomycin C (A). In the Ames test, PADK did not produce mutant colonies, in contrast to a mutagen tested in parallel (B). PADK-injected C57BL/6 mice (ip daily for 9 days) exhibited a dose-dependent increase in the active cathepsin in brain samples (C), whereas synaptic markers were unchanged (D). Alanine aminotransferase (ALT) and blood urea nitrogen (BUN) measures in plasma were also unchanged (E), indicating no effect on major organs. Prior behavioral tests found no adverse effects on the motor coordination rotarod test or on spatial memory using conditioned place preference (F). Organ sections were H+E stained and tissue integrity was evaluated by a pathologist that was blinded to the treatment groups (G).

### DETAILED DESCRIPTION OF THE INVENTION

The present invention provides method for treating a subject afflicted with i) Mild Cognitive Impairment (MCI), ii) early dementia, Iii) early α-synucleinopathy, iv) traumatic brain injury, v) cardiomyopathy, vi) eye disease, or vii) skin damage, comprising administering to the subject at least one compound that increases the level of active cathepsin B in cells of the subject, or a pharmaceutically acceptable salt or ester thereof, in an amount that is effective to treat the subject.

The present invention provides methods for treating a subject afflicted with i) Mild Cognitive Impairment (MCI) or ii) early dementia, comprising administering to the subject at least one compound that increases the level of active cathepsin B in brain cells of the subject, or a pharmaceutically acceptable salt or ester thereof, in an amount that is effective to treat the subject.

In some embodiments, the subject is afflicted with MCI and a symptom of MCI in the subject is cognitive decline, and wherein treating the subject reduces the cognitive decline.

In some embodiments, the cognitive decline comprises memory loss.

In some embodiments, the subject is afflicted with early dementia.

In some embodiments, there is a reduction in glucose use in the brain of the subject.

In some embodiments, there is a reduction in hippocampal volume in the brain of the subject.

In some embodiments, there is decreased expression of synaptophysin, AMPA-type glutamate receptor subunit GluR1 (also called GluA1), GluR2 (also called GluA2), GluR3 (also called GluA3), or GluR4 (also called GluA4), or at least one synapsin isoform, in the brain of the subject relative to i) a previous level of expression of synaptophysin, the AMPA-type glutamate receptor subunit GluR1, GluR2, GluR3, or GluR4, or the at least one synapsin isoform in the brain of the subject, or ii) the level of expression of synaptophysin, the AMPA-type glutamate receptor subunit GluR1, GluR2, GluR3, or GluR4, or the at least one synapsin isoform in the brains of subjects not afflicted with MCI or early dementia.

In some embodiments, there is decreased expression of two, three or four of AMPA-type glutamate receptor subunit GluR1, GluR2, GluR3 and GluR4 in the brain of the subject relative to i) a previous level of expression of the AMPA-type glutamate receptor subunits in the brain of the subject, or ii) the level of expression of the AMPA-type glutamate receptor subunits in the brains of subjects not afflicted with MCI or early dementia. In some embodiments, there is decreased expression of a) GluR1 and GluR2, b) GluR1 and GluR3, c) GluR1 and GluR4, d) GluR1, GluR2 and GluR3, e) GluR1, GluR2 and GluR4, f) GluR1, GluR3 and GluR4, g) GluR2, GluR3 and GluR4, or h) GluR1, GluR2, GluR3, and GluR4, in the brain of the subject relative to i) a previous level of expression of a) GluR1 and GluR2, b) GluR1 and GluR3, c) GluR1 and GluR4, d) GluR1, GluR2 and GluR3, e) GluR1, GluR2 and GluR4, f) GluR1, GluR3 and GluR4, g) GluR2, GluR3 and GluR4, or h) GluR1, GluR2, GluR3, and GluR4 in the brain of the subject, or ii) the level of expression of a) GluR1 and GluR2, b) GluR1 and GluR3, c) GluR1 and GluR4, d) GluR1, GluR2 and GluR3, e) GluR1, GluR2 and GluR4, f) GluR1, GluR3 and GluR4, g) GluR2, GluR3 and GluR4, or h) GluR1, GluR2, GluR3, and GluR4 in the brains of subjects not afflicted with MCI or early dementia.

In some embodiments, the subject has been diagnosed with MCI or early dementia using a cognitive test, a family history assessment, a blood test, a cerebrospinal fluid test (CSF), a neuronal connectivity scan, white matter integrity analysis, an magnetic resonance imaging (MRI) test of gray matter, an assessment of the microstructural integrity of axons and their surrounding myelin, a glucose metabolism assessment, a cortical thickness analysis, a multimodality biomarker assessment, genomic analysis, a sarcasm detection assessment, or any combination thereof.

In some embodiments, the brain cells are neurons or glial cells.

In some embodiments, the brain cells are neurons.

In some embodiments, at least one compound induces an increase in the level of GluR1 and/or another glutamate receptor subunit in brain cells. In some embodiments, the at least one compound induces an increase in the level of a) GluR1 and GluR2, b) GluR1 and GluR3, c) GluR1 and GluR4, d) GluR1, GluR2 and GluR3, e) GluR1, GluR2 and GluR4, f) GluR1, GluR3 and GluR4, g) GluR2, GluR3 and GluR4, h) GluR1, GluR2, GluR3, and GluR4, i) GluR1, j) GluR2, k) GluR3, or l) GluR4 in brain cells.

In some embodiments, at least one compound induces an increase in the level of GluR1 and/or other glutamate receptor subunits in neurons. In some embodiments, the at least one compound induces an increase in the level of a) GluR1 and GluR2, b) GluR1 and GluR3, c) GluR1 and GluR4, d) GluR1, GluR2 and GluR3, e) GluR1, GluR2 and GluR4, f) GluR1, GluR3 and GluR4, g) GluR2, GluR3 and GluR4, h) GluR1, GluR2, GluR3, and GluR4, i) GluR1, j) GluR2, k) GluR3, or l) GluR4 in neurons.

The present invention provides a method for treating a subject afflicted with cardiomyopathy comprising administering to the subject at least one compound that increases the level of active cathepsin B in heart cells of the subject, or a pharmaceutically acceptable salt or ester thereof, in an amount that is effective to treat the subject.

In some embodiments, a symptom of the cardiomyopathy is peripheral edema, chest pain, an abnormal electrocardiogram, or myocardial dysfunction, and wherein treating the subject reduces the symptom.

In some embodiments, the cardiomyopathy is dilated cardiomyopathy, hypertrophic cardiomyopathy, or desmin-related cardiomyopathy.

In some embodiments, the dilated cardiomyopathy is idiopathic dilated cardiomyopathy.

In some embodiments, the at least one compound is administered in an amount that is effective to reduce the level of a cardiac amyloid oligomer in the heart cells of the subject.

In some embodiments, the cardiac amyloid oligomer is α-B-crystallin or an α-B-crystallin mutant.

In some embodiments, the heart cells are cardiac myocytes, intracoronary smooth muscle cells, intracoronary endothelial cells, or interstitial myofibroblasts.

In some embodiments, the heart cells are cardiac myocytes.

In some embodiments, the at least one compound increases the ratio between active cathepsin B and high molecular weight species of cathepsin B in heart cells.

In some embodiments, the high molecular weight species of cathepsin B has or have a molecular weight of 80-110 kiloDaltons (kDa). In some embodiments, the high molecular weight species of cathepsin B has a molecular weight of about 105 kDa.

The present invention provides a method for treating a subject afflicted with i) a retinal disease, ii) an optic nerve disease, iii) retinal damage, or iv) optic nerve damage, comprising administering to the subject at least one compound that increases the level of active cathepsin B in eye cells of the subject, or a pharmaceutically acceptable salt or ester thereof, in an amount that is effective to treat the subject.

In some embodiments, the subject is afflicted with a retinal disease.

In some embodiments, the retinal disease is ischemic retinopathy, diabetic retinopathy, macular degeneration, or retinitis pigmentosa.

In some embodiments, the retinal disease is macular degeneration.

In some embodiments, the macular degeneration is age-related macular degeneration.

In some embodiments, the age-related macular degeneration is wet age-related macular degeneration.

In some embodiments, the age-related macular degeneration is dry age-related macular degeneration.

In some embodiments, the subject is afflicted with an optic nerve disease.

In some embodiments, the optic nerve disease is optic neuropathy.

In some embodiments, the optic neuropathy is ischemic optic neuropathy or glaucoma.

In some embodiments, the subject is afflicted with retinal damage.

In some embodiments, the retinal damage is iatrogenic retinopathy, a retinal tear, or a retinal hole.

In some embodiments, the at least one compound is administered in an amount that is effective to reduce the level of amyloid β (Aβ) vitronectin, amyloid P, orapolipoprotein E in the eye cells of the subject.

In some embodiments, the eye cells are photoreceptor cells.

In some embodiments, the photoreceptor cells are cone cells.

In some embodiments, the photoreceptor cells are rod cells.

The present invention provides a method for treating a subject afflicted with traumatic brain injury (TBI), comprising administering to the subject at least one compound that increases the level of active cathepsin B in brain cells of the subject, or a pharmaceutically acceptable salt or ester thereof, in an amount that is effective to treat the subject.

In some embodiments, the TBI was sustained by the subject while participating in a contact sport, and wherein the contact sport is boxing, martial arts, mixed martial arts, football, or rugby.

In some embodiments, the TBI is from an explosion shockwave.

In some embodiments, the brain cells are neurons or glial cells.

In some embodiments, the brain cells are neurons, and the at least one compound is administered in an amount that is effective to reduce the level of at least one accumulated protein that is potentially pathogenic from the neurons of the subject.

In some embodiments, the at least one compound is administered in an amount that is effective to reduce the level of at least one accumulated protein that is potentially pathogenic from the axonal bulbs or varicosities of neurons of the subject.

In some embodiments, at least one accumulated protein is neurofilament protein, amyloid precursor protein (APP) or a fragment thereof, beta-site APP cleaving enzyme (BACE), presenilin-1 (PS1), an Abeta (Aβ) peptide of 39-43 amino acids, tau protein, α-synuclein, heparan sulfate proteoglycan, or any combination thereof.

In some embodiments, the α-synuclein is mutant α-synuclein. In some embodiments the α-synuclein mutant is an A53T mutant having amino acids in the sequence set forth as.

In some embodiments, the amyloid precursor protein (APP) or fragment thereof is an APP carboxyterminal fragment (APP-CTF). In some embodiments, the APP-CTF is APP-αCTF or APP-βCTF.

In some embodiments, the Aβ peptide has amino acids in the sequence set forth as SEQ ID NO: 1, 2 or 3.

In some embodiments, the tau protein is phosphorylated. In some embodiments, the tau protein has amino acids in the sequence set forth as SEQ ID NO:6 or 7. In some embodiments, the tau protein has amino acids in the sequence set forth as SEQ ID NO: 6, and is phosphorylated at Ser-214, Ser-548, Ser-554, Ser-579, Ser-602, Ser-606, Ser-610, Ser-622, Ser-641, Ser-669, Ser-673, Ser-713, Ser-717, or Ser-721. In some embodiments, the tau protein has amino acids in the sequence set forth as SEQ ID NO:7, and is phosphorylated at T205, T212, T217, S199, S214, T231, S262, S356, S396, S400, S404, S409, or S422.

The present invention provides a method for treating a subject afflicted with skin damage, comprising administering to the subject at least one compound that increases the level of active cathepsin B in skin or hypodermis cells of the subject, or a pharmaceutically acceptable salt or ester thereof, in an amount that is effective to treat the subject.

In some embodiments, the skin damage is a sunburn.

In some embodiments, the skin damage is dermal photodamage.

In some embodiments, the dermal photodamage is dermal photoaging.

In some embodiments, the dermal photodamage is from solar UV-radiation.

In some embodiments, the UV-radiation is UVA-radiation.

In some embodiments, the skin damage is a skin wound.

In some embodiments, the skin wound is skin laceration.

In some embodiments, the skin wound is a skin puncture.

In some embodiments, the skin wound is a skin abrasion.

In some embodiments, the skin wound is a skin burn.

In some embodiments, the at least one compound increases the level of active cathepsin B in skin cells of the subject.

In some embodiments, the skin cells are keratinocytes, Merkel cells, melanocytes, or Langerhans cells.

In some embodiments, the at least one compound increases the level of active cathepsin B in hypodermis cells of the subject.

In some embodiments, the hypodermis cells are fibroblasts, macrophages, or adipocytes.

In some embodiments, the at least one compound is administered to the subject parenterally, by inhalation, intranasally, topically, subcutaneously, intramuscularly, rectally or by intrapulmonary injection.

In some embodiments, the at least one compound is orally administered to the subject.

In some embodiments, the at least one compound is orally administered to the subject in a pharmaceutically acceptable carrier that has a high oil content or a high protein content.

In some embodiments, the pharmaceutically acceptable carrier has an oil content of about 25, 30, 35, 40, 45, 50, 55, 60, 65, 70, 75, 80, 85, 90, 95, 99, or 100% (w/w).

In some embodiments, the pharmaceutically acceptable carrier has a protein content of about 25, 30, 35, 40, 45, 50, 55, 60, 65, 70, 75, 80, 85, 90, 95, 99, or 100% (w/w).

In some embodiments, the pharmaceutically acceptable carrier comprises animal oil.

In some embodiments, the animal oil is fish oil.

In some embodiments, the pharmaceutically acceptable carrier comprises vegetable oil.

In some embodiments, the pharmaceutically acceptable carrier comprises peanut butter.

In some embodiments, the pharmaceutically acceptable carrier is peanut butter.

In some embodiments, the at least one compound is administered to the subject once, twice, or three times per day.

In some embodiments, when the at least one compound is administered to the subject, it is administered at a dose of about 0.5, 1, 1.5, 2, 2.5, 3, 3.5, 4, 4.5 or 5 mg/kg.

In some embodiments, the at least one compound causes no adverse side effect in the subject.

The present invention provides a method for treating a subject afflicted with a protein accumulation disease or TBI, comprising orally administering to the subject at least one compound that increases the level of active cathepsin B in cells of the subject, or a pharmaceutically acceptable salt or ester thereof, in an amount that is effective to treat the subject.

In some embodiments, the subject is afflicted with a protein accumulation disease.

In some embodiments, the protein accumulation disease is
i) brain protein accumulation disease, and the cells are brain cells;
ii) cardiomyopathy, and the cells are heart cells;
iii) macular degeneration, and the cells are eye cells; or
iv) skin damage, and the cells are skin cells or hypodermis cells.

In some embodiments, the protein accumulation disease is a brain protein accumulation disease and the cells are brain cells.

In some embodiments, the brain protein accumulation disease is Alzheimer's disease, pre-Alzheimer's disease, early Alzheimer's disease, early-onset Alzheimer's disease, late-onset Alzheimer's disease, Huntington's disease, α-synucleinopathy, MCI, dementia, or amyotrophic lateral sclerosis.

In some embodiments, the brain protein accumulation disease is early α-synucleinopathy.

In some embodiments, the α-synucleinopathy is Parkinson's disease.

In some embodiments, the brain protein accumulation disease is MCI.

In some embodiments, the brain protein accumulation disease is dementia.

In some embodiments, the dementia is early dementia.

In some embodiments, the dementia is intermediate dementia.

In some embodiments, the dementia is late dementia.

In some embodiments, the brain protein accumulation disease is Alzheimer's disease.

In some embodiments, the brain protein accumulation disease is Huntington's disease.

In some embodiments, the brain protein accumulation disease is amyotrophic lateral sclerosis.

In some embodiments, the subject has been diagnosed with the brain protein accumulation disease using a cognitive test, a family history assessment, a blood test, a cerebrospinal fluid test (CSF), a neuronal connectivity scan, white matter integrity analysis, an magnetic resonance imaging (MRI) test of gray matter, an assessment of the microstructural integrity of axons and their surrounding myelin, a glucose metabolism assessment, a cortical thickness analysis, a multimodality biomarker assessment, genomic analysis, a sarcasm detection assessment, or any combination thereof.

In some embodiments, the brain cells are neurons or glial cells.

In some embodiments, the brain cells are neurons.

In some embodiments, the at least one compound induces an increase in the level of GluR1, 2, 3, or 4 in brain cells.

In some embodiments, the at least one compound induces an increase in the level of GluR1, 2, 3, or 4 in neurons.

In some embodiments, there is a reduction in glucose use in the brain of the subject.

In some embodiments, hippocampal volume in the brain of the subject is reduced.

In some embodiments, there is decreased expression of synaptophysin, AMPA-type glutamate receptor subunit GluR1, GluR2, GluR3, or GluR4, an acetylcholinesterase (AchE) enzyme isoform or at least one synapsin isoform, in the brain of the subject relative to i) a previous level of expression of synaptophysin, the AMPA-type glutamate receptor subunit GluR1, GluR2, GluR3, or GluR4, or the at least one synapsin isoform in the brain of the subject, or ii) the level of expression of synaptophysin, the AMPA-type glutamate receptor subunit GluR1, GluR2, GluR3, or GluR4, or the at least one synapsin isoform in the brains of subjects not afflicted with the brain protein accumulation disease.

In some embodiments, the subject is afflicted with traumatic brain injury (TBI) and the cells are brain cells.

In some embodiments, the active cathepsin B has a molecular weight of about 25-30 kDa. In some embodiments, the active cathepsin B has a molecular weight of about 25 kDa. In some embodiments, the active cathepsin B has a molecular weight of about 30 kDa. In some embodiments, there is a first and a second active cathepsin B isoform, and the first and second active cathepsin B isoforms have a molecular weight of about 25 kDa and about 30 kDa, respectively.

In some embodiments, the subject is a mammalian subject.

In some embodiments, the mammalian subject is a human subject.

In some embodiments, the human subject is at least about 20, 25, 30, 35, 40, 45, 50, 55, 60, 65, 70, 75, 80, 85 or 90 years of age.

In some embodiments, the at least one compound is orally administered to the subject in a pharmaceutically acceptable carrier.

In some embodiments, the pharmaceutically acceptable carrier has a high oil content.

In some embodiments, the pharmaceutically acceptable carrier has an oil content of about 25, 30, 35, 40, 45, 50, 55, 60, 65, 70, 75, 80, 85, 90, 95, 99, or 100% (w/w).

In some embodiments, the pharmaceutically acceptable carrier has a high protein content.

In some embodiments, the pharmaceutically acceptable carrier has a protein content of about 25, 30, 35, 40, 45, 50, 55, 60, 65, 70, 75, 80, 85, 90, 95, 99, or 100% (w/w).

In some embodiments, the pharmaceutically acceptable carrier comprises animal oil.

In some embodiments, the animal oil is fish oil.

In some embodiments, the pharmaceutically acceptable carrier comprises vegetable oil.

In some embodiments, the pharmaceutically acceptable carrier comprises peanut butter.

In some embodiments, the pharmaceutically acceptable carrier is peanut butter.

In some embodiments, the at least one compound is administered to the subject once, twice, or three times per day.

In some embodiments, when the at least one compound is administered to the subject, it is administered at a dose of about 0.5, 1, 1.5, 2, 2.5, 3, 3.5, 4, 4.5 or 5 mg/kg.

In some embodiments, the at least one compound causes no adverse side effect in the subject.

In some embodiments, at least one compound is Z-phenylalanyl-alanyl-diazomethylketone (PADK) or a PADK analogue, or a pharmaceutically acceptable salt or ester thereof.

In some embodiments, at least one compound is a PADK analogue.

In some embodiments, the PADK analogue is Z-L-phenylalanyl-D-alanyl-diazomethylketone (PdADK) Z-D-phenylalanyl-L-alanyl-diazomethylketone (dPADK) or Z-D-phenylalanyl-D-alanyl-diazomethylketone (dPdADK).

In some embodiments, the PADK analogue is Z-phenylalanyl-phenylalanyl-diazomethylketone (PPDK), or a pharmaceutically acceptable salt or ester thereof.

In some embodiments, the PADK analogue is Z-L-phenylalanyl-D-phenylalanyl-diazomethylketone (PdPDK) Z-D-phenylalanyl-L-phenylalanyl-diazomethylketone (dPPDK) or Z-D-phenylalanyl-D-phenylalanyl-diazomethylketone (dPdPDK).

In some embodiments, the first or second amino acid chiral carbon of the PADK analogue is methylated, or wherein both the first and second amino acid chiral carbon of the PADK analogue is methylated.

In some embodiments, the PADK analogue has the structure:

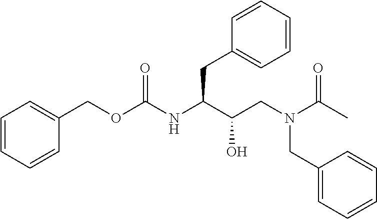 [SMILES: CC(=O)N(Cc1ccccc1)C[C@H](O)[C@H](Cc1ccccc1)NC(=O)OCc1ccccc1]

or a pharmaceutically acceptable salt or ester thereof.

In some embodiments, the PADK analogue has the structure:

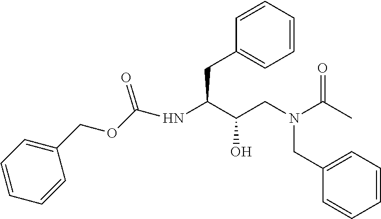 [SMILES: CC(=O)N(Cc1ccccc1)C[C@H](O)[C@H](Cc1ccccc1)NC(=O)OCc1ccccc1]

or a pharmaceutically acceptable salt or ester thereof.

In some embodiments, the PADK analogue has the structure:

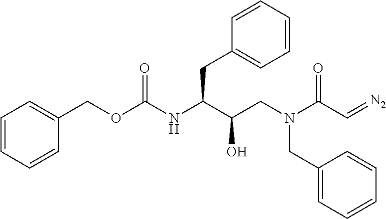

or a pharmaceutically acceptable salt or ester thereof.

In some embodiments, the at least one compound is a lysosomal modulator having the structure:

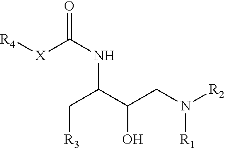

wherein R₁, R₂, R₃, and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring; and
wherein substituted means having one or more atoms replaced with one or more identical or different substituents selected from the group consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkylthio, alkenylthio, alkynylthio, sulfonyl, alkyl sulfonyl, sulfinyl, and alkylsulfinyl,
wherein X is a heteroatom including but not limited to O, N, or S,
or a pharmaceutically acceptable salt or ester thereof.

In some embodiments, the lysosomal modulator has the structure:

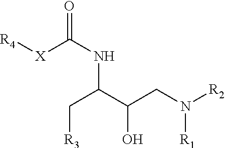

wherein R₁ and R₂ independently are halogen; hydroxyl; alkoxy; acyl; cyano; nitro; amino; substituted or unsubstituted alkyl, alkenyl, or alkynyl groups; or substituted or unsubstituted aromatic or cyclic aliphatic groups, which may include one or more heteroatoms in the ring;
wherein, R₃ and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring;
wherein substituted means substituted with one or more identical or different substituents selected from the group
   consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkyl thio, alkenylthio, alkynylthio, sulfonyl, alkylsulfonyl, sulfinyl, and alkylsulfinyl; and
wherein X is a heteroatom selected from O, N, or S, or a pharmaceutically acceptable salt or ester thereof.

In some embodiments, R₁ and R₂ are independently acyl or substituted or unsubstituted aromatic, heteroaromatic, cyclic aliphatic, or heterocyclic aliphatic rings, and R₃ and R₄ are independently hydrogen; acyl; or substituted or unsubstituted aromatic, heteroaromatic, cyclic aliphatic, or heterocyclic aliphatic rings.

In some embodiments, R₁, R₃, and R₄ independently are substituted or unsubstituted phenyl, benzyl, or napthylmethyl; and wherein R₂ is selected from the group consisting of acetyl, diazoacetyl, and benzoyl groups.

In some embodiments, the lysosomal modulator is a diastereomerically pure compound wherein the stereocenters have the configuration RR, RS, SR, or SS.

In some embodiments, at least one compound is leupeptin, deacetyl-leupeptin, E-64, E-64c, E-64d, diazoacetyl-DL-2-aminohexanoic acid methyl ester, bafilomycin A1, glycyl-phenylalanyl-glycine-aldehyde semicarbazone, or pepstatin A, or a pharmaceutically acceptable salt or ester thereof.

In some embodiments, at least one compound is a combination of two, three, four, or more of:
i) PADK or a PADK analogue, or a pharmaceutically acceptable salt or ester thereof;
ii) a lysosomal modulator having the structure:

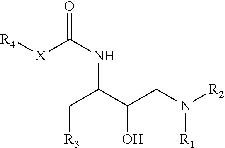

wherein R₁ and R₂ independently are halogen; hydroxyl; alkoxy; acyl; cyano; nitro; amino; substituted or unsubstituted alkyl, alkenyl, or alkynyl groups; or substituted or unsubstituted aromatic or cyclic aliphatic groups, which may include one or more heteroatoms in the ring;
wherein, R₃ and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring;
wherein substituted means substituted with one or more identical or different substituents selected from the group consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkyl thio, alkenylthio, alkynylthio, sulfonyl, alkylsulfonyl, sulfinyl, and alkylsulfinyl; and
wherein X is a heteroatom selected from O, N, or S, or a pharmaceutically acceptable salt or ester thereof;
iii) PPDK or a pharmaceutically acceptable salt or ester thereof;
iv) a PADK analogue having the structure:

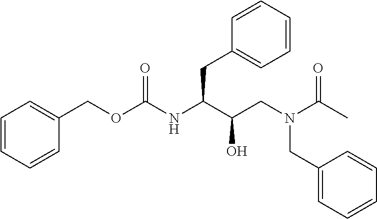 [SMILES: CC(=O)N(Cc1ccccc1)C[C@@H](O)[C@H](Cc1ccccc1)NC(=O)OCc1ccccc1]

or a pharmaceutically acceptable salt or ester thereof;
v) a PADK analogue having the structure:

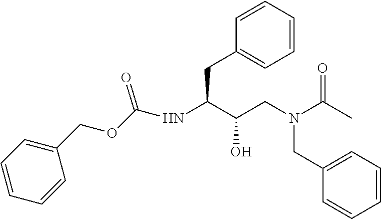 [SMILES: CC(=O)N(Cc1ccccc1)C[C@H](O)[C@H](Cc1ccccc1)NC(=O)OCc1ccccc1]

or a pharmaceutically acceptable salt or ester thereof;
vi) a PADK analogue having the structure:

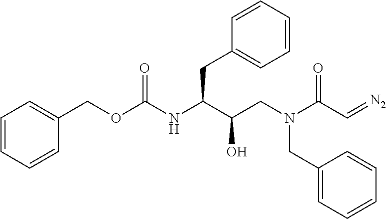

or a pharmaceutically acceptable salt or ester thereof;
vii) leupeptin or a pharmaceutically acceptable salt or ester thereof;
viii) deacetyl-leupeptin or a pharmaceutically acceptable salt or ester thereof;
ix) E-64 or a pharmaceutically acceptable salt or ester thereof;
x) E-64c or a pharmaceutically acceptable salt or ester thereof;
xi) E-64d or a pharmaceutically acceptable salt or ester thereof;
xii) diazoacetyl-DL-2-aminohexanoic acid methyl ester or a pharmaceutically acceptable salt or ester thereof;
xiii) bafilomycin A1 or a pharmaceutically acceptable salt or ester thereof;
xiv) glycyl-phenylalanyl-glycine-aldehyde semicarbazone or a pharmaceutically acceptable salt or ester thereof; or
xv) pepstatin A or a pharmaceutically acceptable salt or ester thereof,
each in an amount that when administered together is effective to treat the subject, or increase the level of active cathepsin B in brain, heart, eye, or skin cells of the subject.

In some embodiments, at least one compound increases the level of active cathepsin B in brain, heart, eye, or skin cells at least about 1.5, 2, 3, 4, 5, 6, 7, 8, 9, 10, 11, 12, 14, 14, or 15-fold.

In some embodiments, at least one compound increases cathepsin B trafficking in brain, heart, eye, or skin cells.

In some embodiments, at least one compound increases cathepsin B maturation in brain, heart, eye, or skin cells.

A composition for treating a subject afflicted with a protein accumulation disease or TBI, comprising at least one compound disclosed herein, and a pharmaceutically acceptable carrier.

In some embodiments, at least one compound is Z-phenylalanyl-alanyl-diazomethylketone (PADK) or a PADK analogue, or a pharmaceutically acceptable salt or ester thereof. PADK may also be referred to as Z-Phe-Ala-diazomethylketone. The CAS Number for PADK is 71732-53-1. PADK is commercially available from Bachem Americas, Inc. (N-1040; Torrance, Calif., USA).

In some embodiments, at least one compound is a lysosomal modulator having the structure:

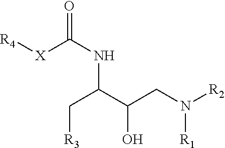

wherein R₁, R₂, R₃, and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring; and
wherein substituted means having one or more atoms replaced with one or more identical or different substituents selected from the group consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkylthio, alkenylthio, alkynylthio, sulfonyl, alkyl sulfonyl, sulfinyl, and alkylsulfinyl,
wherein X is a heteroatom including but not limited to O, N, or S,
   or a pharmaceutically acceptable salt or ester thereof.

Compounds of this structure, as well as processes of synthesizing compounds of this structure are described in U.S. Pat. No. 8,163,953, the entire contents of which are incorporated herein by reference.

In some embodiments, the at least one compound is a lysosomal modulator having the structure:

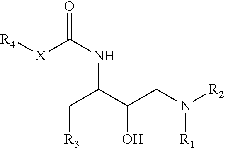

wherein R₁ and R₂ independently are halogen; hydroxyl; alkoxy; acyl; cyano; nitro; amino; substituted or unsubstituted alkyl, alkenyl, or alkynyl groups; or substituted or unsubstituted aromatic or cyclic aliphatic groups, which may include one or more heteroatoms in the ring;
wherein, R₃ and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring;
wherein substituted means substituted with one or more identical or different substituents selected from the group consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkyl thio, alkenylthio, alkynylthio, sulfonyl, alkylsulfonyl, sulfinyl, and alkylsulfinyl; and
wherein X is a heteroatom selected from O, N, or S, or a pharmaceutically acceptable salt or ester thereof.

Compounds of this structure, as well as processes of synthesizing compounds of this structure are described in U.S. Pat. No. 8,163,953, the entire contents of which are incorporated herein by reference.

In some embodiments, the PADK analogue is Z-phenylalanyl-phenylalanyl-diazomethylketone (PPDK), or a pharmaceutically acceptable salt or ester thereof. PPDK is also known as Z-Phe-Phe-diazomethylketone. The CAS Number for PPDK is 65178-14-5. PPDK is commercially available from Sachem Americas, Inc. (N-1045; Torrance, Calif., USA).

The structures for PADK and PPDK are:

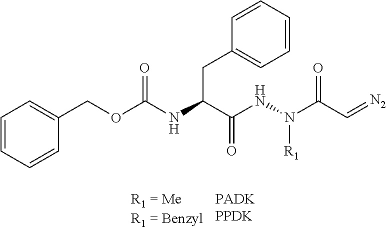

The structure for PADK is:

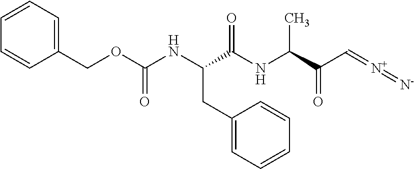 [SMILES: C[C@H](NC(=O)[C@H](Cc1ccccc1)NC(=O)OCc1ccccc1)C(=O)C=[N+]=[N-]]

The structure for PPDK is:

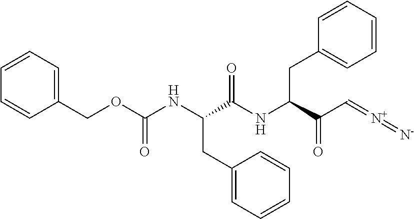 [SMILES: [N-]=[N+]=CC(=O)[C@H](Cc1ccccc1)NC(=O)[C@H](Cc1ccccc1)NC(=O)OCc1ccccc1]

In some embodiments, the at least one compound has the structure:

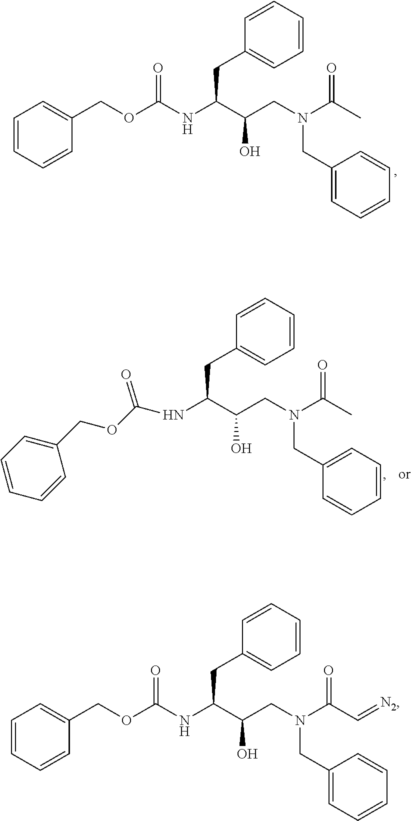

or a pharmaceutically acceptable salt or ester thereof. Methods for synthesizing these compounds are described in Viswanathan et al (2012) “Nonpeptidic Lysosomal Modulators Derived from Z-Phe-Ala-Diazomethylketone for Treating Protein Accumulation Diseases” ACS Med. Chem. Lett., 3, 920-924, the entire contents of which are incorporated herein by reference. Additionally, Synthesis Schemes for these compounds are provided below.

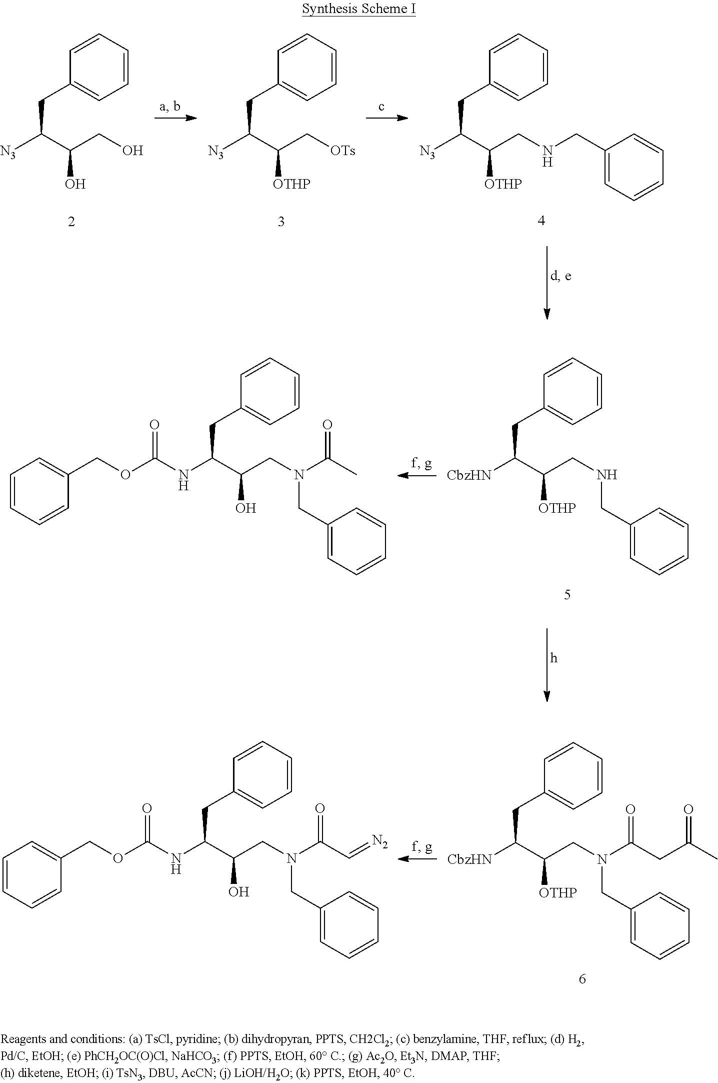

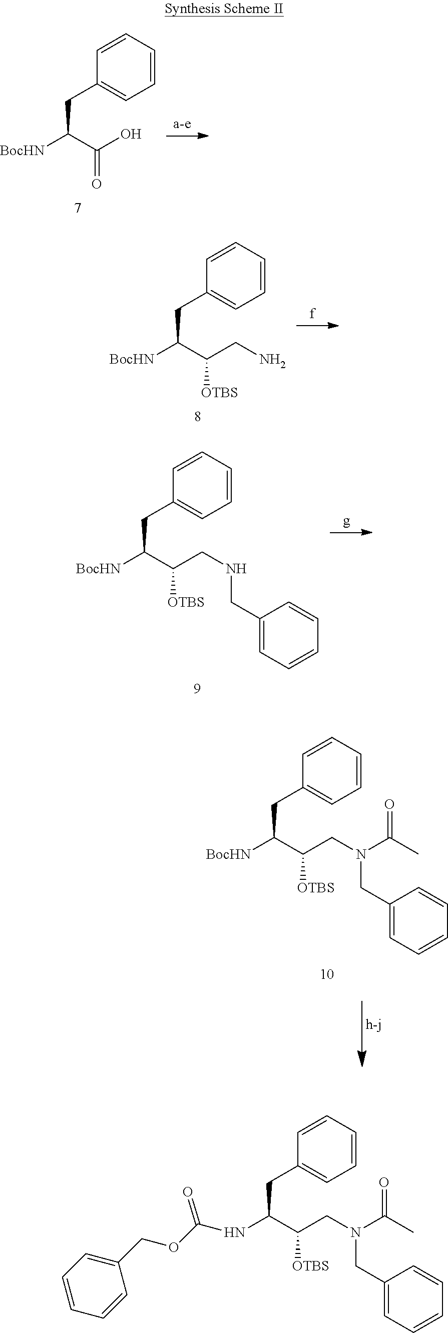 [SMILES: CC(C)(C)OC(=O)N[C@@H](Cc1ccccc1)C(=O)O ; CC(C)(C)OC(=O)N[C@@H](Cc1ccccc1)[C@H](CN)O[Si](C)(C)C(C)(C)C ; CC(C)(C)OC(=O)N[C@@H](Cc1ccccc1)[C@H](CNCc1ccccc1)O[Si](C)(C)C(C)(C)C ; CC(=O)N(Cc1ccccc1)C[C@H](O[Si](C)(C)C(C)(C)C)[C@H](Cc1ccccc1)NC(=O)OC(C)(C)C ; CC(=O)N(Cc1ccccc1)C[C@H](O[Si](C)(C)C(C)(C)C)[C@H](Cc1ccccc1)NC(=O)OCc1ccccc1]

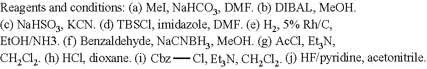 [SMILES: O=C(Cl)OCc1ccccc1]

In some embodiments, the PADK analogue is Z-L-phenylalanyl-D-alanyl-diazomethylketone (PdADK) Z-D-phenylalanyl-L-alanyl-diazomethylketone (dPADK) or Z-D-phenylalanyl-D-alanyl-diazomethylketone (dPdADK). These compounds may be obtained from Bachem Americas Inc., 3132 Kashiwa Street, Torrance, Calif. 90505, USA (Quotation 0007502513).

In some embodiments, at least one compound is leupeptin or a pharmaceutically acceptable salt or ester thereof. The CAS Number for leupeptin is 55123-66-5. The Chemspider Number for leupeptin is 65357. Leupeptin is also known as N-Acetyl-L-leucyl-N-{(2S)-5-[(diaminomethylene)amino]-1-oxo-2-pentanyl}-L-leucinamide and N-acetyl-L-leucyl-L-leucyl-L-argininal. Leupeptin is commercially available from Affymetrix, (18413, Santa Clara, Calif., USA).

In some embodiments, at least one compound is deacetyl-leupeptin or a pharmaceutically acceptable salt or ester thereof. The CAS Number for deacetyl-leupeptin is 81344-47-0. The PubChem Substance ID for deacetyl-leupeptin is 24892002. Deacetyl-leupeptin is also known as α-Boc-Deacetylleupeptin, and is commercially available from Sigma-Aldrich (B7530, St. Louis, Mo., USA). The structure of deacetyl-leupeptin is:

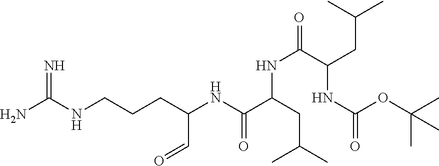 [SMILES: CC(C)CC(NC(=O)OC(C)(C)C)C(=O)NC(CC(C)C)C(=O)NC(C=O)CCCNC(=N)N]

In some embodiments, at least one compound is E-64 or a pharmaceutically acceptable salt or ester thereof. The CAS Number for E-64 is 66701-25-5. The PubChem Substance ID for E-64 is 24278411. E-64 is commercially available from Sigma-Aldrich (E3132, St. Louis, Mo., USA). The structure of E-64 is:

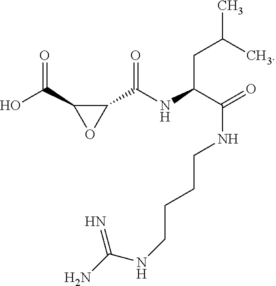 [SMILES: CC(C)C[C@H](NC(=O)[C@@H]1O[C@H]1C(=O)O)C(=O)NCCCCNC(=N)N]

In some embodiments, at least one compound is E-64c or a pharmaceutically acceptable salt or ester thereof. The CAS Number for E-64c is 76684-89-4. The PubChem Substance ID for E-64c is 24894369. E-64c is commercially available from Sigma-Aldrich (E0514, St. Louis, Mo., USA). The structure of E-64c is:

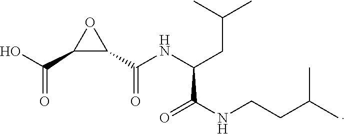 [SMILES: CC(C)CCNC(=O)[C@H](CC(C)C)NC(=O)[C@H]1O[C@@H]1C(=O)O]

In some embodiments, at least one compound is E-64d or a pharmaceutically acceptable salt or ester thereof. The CAS Number for E-64d is 88321-09-9. The PubChem Substance ID for E-64d is 24894681. E-64d is commercially available from Sigma-Aldrich (E8640, St. Louis, Mo., USA). The structure of E-64d is:

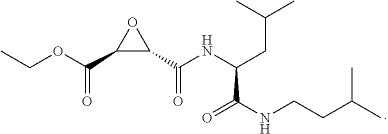 [SMILES: CCOC(=O)[C@H]1O[C@@H]1C(=O)N[C@@H](CC(C)C)C(=O)NCCC(C)C]

In some embodiments, at least one compound is diazoacetyl-DL-2-aminohexanoic acid methyl ester or a pharmaceutically acceptable salt or ester thereof. The CAS Number for diazoacetyl-DL-2-aminohexanoic acid methyl ester is 7013-09-4. Diazoacetyl-DL-2-aminohexanoic acid methyl ester is commercially available from Sachem Americas, Inc. (F-2220; Torrance, Calif., USA). The structure of diazoacetyl-DL-2-aminohexanoic acid methyl ester is:

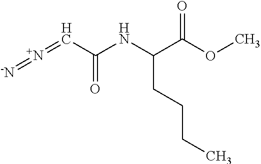

In some embodiments, at least one compound is bafilomycin A1 or a pharmaceutically acceptable salt or ester thereof. The CAS Number for bafilomycin A1 is 88899-55-2. The PubChem Substance ID for bafilomycin A1 is 24891613. The ChemSpider ID for bafilomycin A1 is 10251049. Bafilomycin A1 is commercially available from Sigma-Aldrich (81793, St. Louis, Mo., USA). The structure of bafilomycin A1 is:

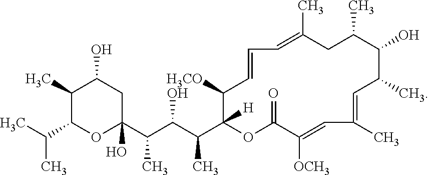 [SMILES: CO/C1=C\C(C)=C\[C@@H](C)[C@@H](O)[C@@H](C)C/C(C)=C/C=C/[C@H](OC)[C@@H]([C@@H](C)[C@@H](O)[C@H](C)[C@@]2(O)C[C@@H](O)[C@H](C)[C@@H](C(C)C)O2)OC1=O]

In some embodiments, at least one compound is glycyl-phenylalanyl-glycine-aldehyde semicarbazone or a pharmaceutically acceptable salt or ester thereof. Glycyl-phenylalanyl-glycine-aldehyde semicarbazone is also known as H-Gly-Phe-Gly-aldehyde semicarbazone and glycyl-phenylalanyl-glycylsemicarbazone. Glycyl-phenylalanyl-glycine-aldehyde semicarbazone is commercially available from Bachem Americas, Inc. (H-7650, Torrance, Calif., USA). The structure of H-Gly-Phe-Gly-aldehyde semicarbazone is:

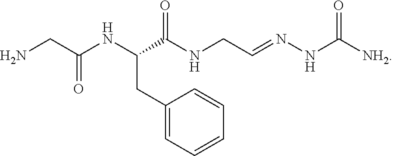 [SMILES: NCC(=O)N[C@@H](Cc1ccccc1)C(=O)NC/C=N/NC(N)=O]

In some embodiments, at least one compound is pepstatin A or a pharmaceutically acceptable salt or ester thereof. The CAS Number for pepstatin A is 26305-03-3. The PubChem Substance ID for pepstatin A is 24898620. The ChemSpider ID for pepstatin A is 4586014. Pepstatin A is commercially available from Sigma-Aldrich (P5318, St. Louis, Mo., USA). The structure of pepstatin A is:

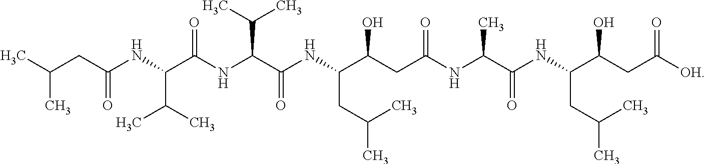 [SMILES: CC(C)CC(=O)N[C@H](C(=O)N[C@H](C(=O)N[C@@H](CC(C)C)[C@@H](O)CC(=O)N[C@@H](C)C(=O)N[C@@H](CC(C)C)[C@@H](O)CC(=O)O)C(C)C)C(C)C]

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### Key to the Sequence Listing

### SEQ ID NO: 1 Beta-Amyloid (1-42), Human

### SEQ ID NO: 2 Beta-Amyloid (1-43), Human

### SEQ ID NO: 3 Beta-Amyloid (1-40), Human

### SEQ ID NO: 4 APP-αCTF, Human

### SEQ ID NO: 5 APP-βCTF, Human

SEQ ID NO: 6 A full-length human Tau isoform
SEQ ID NO: 7 A spliced variant of human Tau
SEQ ID NO: 8 α-synuclein, Human (A53T mutation)

### Terms

Unless otherwise defined, all technical and scientific terms used herein have the same meaning as commonly understood by a person of ordinary skill in the art to which this invention belongs.

As used herein, and unless stated otherwise or required otherwise by context, each of the following terms shall have the definition set forth below.

As used herein, “about” in the context of a numerical value or range means ±10% of the numerical value or range recited or claimed, unless the context requires a more limited range.

As used herein, “treating” means slowing, stopping, or reversing the progression of a disease.

### Protein Accumulation Diseases

As used herein “protein accumulation diseases” are diseases that are associated with protein accumulation and aggregation.

“Brain protein accumulation diseases” are associated with protein accumulation and aggregation in the brain. For example, Alzheimer's disease involves accumulation of Aβ peptides and tau, Parkinson's disease involves accumulation of α-synuclein, Huntington's disease involves aggregation of mutated huntingtin protein, and amyotrophic lateral sclerosis involves accumulation of mutated superoxide dismutase-1 protein. Some chronic psychiatric disorders, such as schizophrenia, bipolar disorder, and recurrent major depression, have also been associated with protein aggregation. The reduction of protein accumulation events is important for slowing the progression of these diseases and disorders. Studies indicate that protein degradation processes that clear these aggregated proteins could provide treatment for some or all of these diseases and disorders.

As used herein, “Mild Cognitive Impairment” or “MCI” means an impairment in memory that is not severe enough to affect daily function and that is linked to a condition other than Alzheimer's disease, other than Parkinson's disease, and other than Huntington's disease. MCI may comprise deficits in cognitive functions in addition to impaired memory. Subjects with MCI have actual memory loss, rather than the sometimes slow memory retrieval from relatively preserved memory storage in age-matched controls. MCI tends to first affect short-term (also called episodic) memory first. Subjects have trouble remembering recent conversations, the location of commonly used items, and appointments. However, memory for remote events is typically intact, as is attention (also called working memory-subjects can repeat lists of items and do simple calculations). MCI often progresses to dementia.

MCI is an aspect of cognitive aging that is also considered to be a transitional state between normal aging and the dementia into which it may convert. MCI has received increasing acceptance as a distinct stage between normal cognitive aging and dementia (See, e.g., Mariani et al., 2007; Petersen et al., 2009, the entire contents of each of which are hereby incorporated herein by reference). Regarding aspects of MCI, the reductions in the brain's use of glucose (i.e. glucose metabolism linked to neuronal activity) and hippocampal volume are often detected 10-15 years before the first sign of cognitive decline or memory loss (Bateman et al., 2012). The causes of MCI are not yet completely understood. Experts believe that many cases—but not all—result from brain changes occurring in the very early stages of Alzheimer's disease or other dementias, thus MCI-related brain changes are not restricted to being linked to Alzheimer's disease (AD) (see Fjell et al., 2013). The reduced brain function of MCI is a nuisance, and it can lead to significant impairment of a person's ability to learn new information, solve problems, or carry out everyday activities. In some embodiments of the invention, the progression of one or more, or all of the symptoms of MCI discussed above is slowed or reversed.

MCI causes greater memory loss than age-associated memory impairment; memory and sometimes other cognitive functions are worse in subjects with this disorder than in age-matched controls, but daily functioning is not affected. In contrast, dementia impairs daily functioning. Up to 50% of subjects with mild cognitive impairment develop dementia within 3 years. See, e.g., The Merk Manual, “Dementia” full review/revision April 2013 by Juebin Huang, MD, PhD, available from www.merckmanuals.com/professional/neurologic_disorders/delirium_and_dementia/dementia.html, the entire contents of which are hereby incorporated herein by reference. MCI and early dementia are described in Fleisher AS1, Chen K, Quiroz Y T, Jakimovich L J, Gomez M G, Langois C M, Langbaum J B, Ayutyanont N, Roontiva A, Thiyyagura P, Lee W, Mo H, Lopez L, Moreno S, Acosta-Baena N, Giraldo M, Garcia G, Reiman R A, Huentelman M J, Kosik K S, Tariot P N, Lopera F, Reiman E M. Florbetapir PET analysis of amyloid-β deposition in the presenilin 1 E280A autosomal dominant Alzheimer's disease kindred: a cross-sectional study. Lancet Neurol. 2012 11:1057-65; and in Fjell A M, Walhovd K B, Fennema-Notestine C, McEvoy L K, Hagler D J, Holland D, Brewer J B, Dale A M; Alzheimer's Disease Neuroimaging Initiative. CSF biomarkers in prediction of cerebral and clinical change in mild cognitive impairment and Alzheimer's disease. J Neurosci. 2010 Feb. 10; 30(6):2088-101, the entire content of each of which is hereby incorporated herein by reference.

As used herein, “dementia” means memory loss plus evidence of cognitive and behavioral dysfunction. A subject with dementia may have difficulty with finding words and/or naming objects (aphasia), doing previously learned motor activities (apraxia), or planning and organizing everyday tasks, such as meals, shopping, and bill paying (impaired executive function). A subject's personality may change; for example, the subject may become uncharacteristically irritable, anxious, agitated, and/or inflexible.

Clinical criteria for dementia include cognitive or behavioral (neuropsychiatric) symptoms that interfere with the ability to function at work or do usual daily activities. These symptoms represent a decline from previous levels of functioning. These symptoms are not explained by delirium or a major psychiatric disorder. In some embodiments, the cognitive or behavioral impairment involves ≧2 of the following domains: i) Impaired ability to acquire and remember new information (amnesia), ii) Language dysfunction (aphasia), iii) Visuospatial dysfunction (agnosia; eg, inability to recognize faces or common objects), iv) Impaired executive function, including reasoning, handling of complex tasks, and/or judgment (apraxia); v) Changes in personality, behavior, or comportment.

Dementia impairs cognition globally. Onset is gradual, although family members may suddenly notice deficits (e.g., when function becomes impaired). Often, loss of short-term memory is the first sign. Although symptoms of dementia exist in a continuum, they can be divided into early, intermediate, and late. Personality changes and behavioral disturbances may develop early or late. Motor and other focal neurologic deficits occur at different stages, depending on the type of dementia. Incidence of seizures is somewhat increased during all stages. Psychosis—hallucinations, delusions, or paranoia—occurs in about 10% of subjects with dementia, although a higher percentage may experience these symptoms temporarily.

During “early dementia” Recent memory is impaired; learning and retaining new information become difficult. Language problems (especially with word finding), mood swings, and personality changes develop. Subjects may have progressive difficulty with independent activities of daily living (e.g., balancing their checkbook, finding their way around, remembering where they put things). Abstract thinking, insight, or judgment may be impaired. Subjects may respond to loss of independence and memory with irritability, hostility, and agitation. Functional ability may be further limited by the following:

Agnosia: Impaired ability to identify objects despite intact sensory function.
Apraxia: Impaired ability to do previously learned motor activities despite intact motor function. Aphasia: Impaired ability to comprehend or use language.

Although early dementia may not compromise sociability, family members may report strange behavior accompanied by emotional lability. In embodiments of the invention, one, two, three, four, five, or more, or all of the symptoms of early dementia discussed above are reduced, reversed, or prevented. In some embodiments of the invention, the progression of one, two, three, four, five, or more, or all of the symptoms of early dementia discussed above is slowed or reversed.

In some embodiments, early dementia is linked to a condition other than AD, other than Parkinson's disease, and other than Huntington's disease.

During “intermediate dementia” subjects become unable to learn and recall new information. Memory of remote events is reduced but not totally lost. Subjects may require help with basic activities of daily living (eg, bathing, eating, dressing, toileting). Personality changes may progress. Subjects may become irritable, anxious, self-centered, inflexible, or angry more easily, or they may become more passive, with a flat affect, depression, indecisiveness, lack of spontaneity, or general withdrawal from social situations. Behavior disorders may develop: subjects may wander or become suddenly and inappropriately agitated, hostile, uncooperative, or physically aggressive.

By this stage, subjects have lost all sense of time and place because they cannot effectively use normal environmental and social cues. Subjects often get lost; they may be unable to find their own bedroom or bathroom. They often remain ambulatory but are at risk of falls or accidents secondary to confusion. Altered sensation or perception may culminate in psychosis with hallucinations and paranoid and persecutory delusions. Sleep patterns are often disorganized. In embodiments of the invention, one, two, three, four, five, or more, or all of the symptoms of intermediate dementia discussed above are reduced, reversed, or prevented. In some embodiments of the invention, the progression of one, two, three, four, five, or more, or all of the symptoms of intermediate dementia discussed above is slowed or reversed.

During “late dementia” subjects cannot walk, feed themselves, or do any other activities of daily living; they may become incontinent. Recent and remote memory is completely lost. Subjects may be unable to swallow. They are at risk of undernutrition, pneumonia (especially due to aspiration), and pressure ulcers. Because they depend completely on others for care, placement in a long-term care facility often becomes necessary. Eventually, subjects become mute. In embodiments of the invention, one, two, three, four, five, or more, or all of the symptoms of late dementia discussed above are reduced, reversed, or prevented. In some embodiments of the invention, the progression of one, two, three, four, five, or more, or all of the symptoms of late dementia discussed above is slowed or reversed.

End-stage dementia results in coma and death, usually due to infection. Cytoplasmic deposition of α-synuclein is a common pathological feature of many neurodegenerative diseases collectively called α-synucleinopathies, including familial Parkinson's disease.

Parkinson's disease (Parkinson's) is a motor system disorder which is associated with the loss of dopamine-producing brain cells. Dopamine is necessary for coordinated muscle function and movement. Dopamine is normally produced by certain nerve cells (neurons) in the substantia nigra region of the brain: however, Parkinson's subjects experience a loss of these neurons which leads to impaired movement. This loss of neurons is associated with the accumulation of a synuclein, a protein that is mutated and/or misfolded in Parkinson's and other diseases. The α-synuclein forms aggregates that accumulate in Lewy bodies, and which are seen in the brains of subjects who have died from Parkinson's.

The age-related neurodegenerative disorder Alzheimer's disease (AD) involves the accumulation of oligomeric peptide species, protein aggregation, and altered brain function. One of the major hallmarks of AD is the plaque deposits consisting primarily of amyloid fibrils formed by the amyloid beta peptide Aβ1-42, as well as the intracellular and extracellular buildup of soluble oligomers of this peptide. Mutations associated with familial AD, including mutations in the amyloid precursor protein (APP), strongly implicate Aβ1-42 as a causative factor since the mutations increase the relative amount of this Aβ peptide. Increased Aβ is one of the earliest events in AD, and, besides extracellular accumulation, Aβ oligomerization also occurs intraneuronally. Aβ oligomers disrupt synaptic plasticity, impair synaptic responses and memory, and cause cytotoxicity, as well as produce synaptic deterioration. Aβ oligomers, especially trimers and multiples of trimeric species, are particularly stable.

There are no current treatments to reduce the abnormal protein accumulation events in AD. Only two classes of drugs are approved for treating AD, acetyl-cholinesterase inhibitors and N-methyl-D-aspartic acid (NMDA) receptor antagonists. Both types of drugs only affect the symptoms of AD. Acetyl-cholinesterase inhibitors are for mild to moderate AD and have modest effects in a small percentage of subjects who take the drug, and are typically ineffective after 6-12 months of use. The NMDA receptor antagonist that is available treats the secondary pathology but not the protein accumulation in mild to severe AD.

The neurodegenerative disorder Huntington's disease (Huntington's) is caused by a trinucleotide repeat expansion in the huntingtin gene which codes for huntingtin protein, “Htt.” People who have Huntington's Disease have more C-A-G codons on their huntingtin gene which results in Htts that are “altered” or abnormal in that they have an excess number of glutamines. As a result of the excess glutamines, these altered Htts form protein aggregates which can interfere with nerve cell function.

Amyotrophic lateral sclerosis (ALS) is a progressive neurodegenerative disease that attacks nerve cells in the brain and the spinal cord. Neuronal cell death has been linked to the presence of aggregates of mutant superoxide dismutase-1 (SOD1) protein. Mutant SOD1 accumulates to form high molecular weight amorphous aggregates which can interact with other proteins. When these mutant SOD1 proteins accumulate and form aggregates in a neuronal cell, the cell almost always dies.

Aspects of the present invention relate to the discovery that PADK and other compounds of the invention increase cathepsin B in eye tissue. Some embodiments of the invention are useful for treating treating retinal or optic nerve disease or damage in subjects.

Age-related macular degeneration (AMD) is the most common cause of irreversible vision loss in the elderly. Age-related macular degeneration (AMD) is characterized by the presence of drusen, which are extracellular deposits that accumulate beneath the retinal pigmented epithelium. Many protein and lipid constituents of drusen are similar to those found in deposits characteristic of other age-related degenerative disorders such as Alzheimer disease (AD) and other amyloid diseases. These include amyloid β (Aβ), vitronectin, amyloid P, apolipoprotein E, and inflammatory mediators such as acute phase reactants and complement components (Isas et al 2010), AMD may be dry or wet AMD.

Dry AMD causes the tissues of the macula to thin as cells disappear. Accumulated waste products from the rods and cones may produce deposits in the retina (the transparent, light-sensitive structure at the back of the eye) called drusen (yellow spots). Both eyes may be affected simultaneously in the dry form. There is no evidence of scarring or of bleeding or other fluid leakage in the macula. In dry AMD, the loss of central vision occurs slowly and painlessly over years. Subjects may have few or no symptoms but, when they do have symptoms, they often occur in both eyes. Objects may appear washed out, fine detail may be lost, and reading may become more difficult. As the disease progresses, central blind spots (scotomas) usually occur and can sometimes severely impair vision. Most subjects retain enough vision to read and drive. In some embodiments, the progression of one, two, three, four, five, or more, or all of the symptoms of dry AMD discussed above is slowed or reversed.

Wet AMD can result from dry AMD. AMD always begins as dry AMD. Some subjects develop wet AMD as well when abnormal blood vessels grow in from the choroid (the layer of blood vessels that lies between the retina and the outer white layer of the eye called the sclera) under the macula and leak blood and fluid (hence the description as “wet”). Eventually, a mound of scar tissue develops under the macula. The wet form develops in one eye first but eventually may affect both eyes. In wet AMD, loss of vision tends to progress quickly, usually over days or weeks, and may be even more sudden if one of the abnormal blood vessels bleeds. The first symptom may be an area of blurry, wavy, or distorted central vision. Vision at the outer edges of the visual field (peripheral vision) is typically not affected. Wet AMD usually affects one eye at a time. Often, difficulty with reading or watching television results. In some embodiments of the invention, the progression of one two, three, four, five, or more, or all of the symptoms of wet AMD discussed above is slowed or reversed. See, e.g., The Merk Manual, “Age-Related Macular Degeneration” full review/revision April 2013 by Juebin Huang, MD, PhD, available from www.merckmanuals.com/home/eye_disorders/retinal_disorders/age-related_macular_degeneration.html, the entire contents of which are hereby incorporated herein by reference.

As used herein, “an amount that is effective to treat the subject” or “therapeutically effective amount” when referring to an amount of a compound refers to the quantity of the compound that is sufficient to yield a desired therapeutic response without undue adverse side effects (such as toxicity, irritation, or allergic response) commensurate with a reasonable benefit/risk ratio when used in the manner of this invention. It will be understood that the specific effective amount will vary with such factors as the particular condition being treated, the physical condition of the subject, the type of subject being treated, the duration of the treatment, the nature of concurrent therapy (if any), and the specific formulations employed and the structure of the compound or its derivatives.

Non-limiting examples of diagnostic tests that are useful in embodiments of the invention are a cognitive test, a family history assessment, a blood test, a cerebrospinal fluid test (CSF), a neuronal connectivity scan, white matter integrity analysis, an magnetic resonance imaging (MRI) test of gray matter, an assessment of the microstructural integrity of axons and their surrounding myelin, a glucose metabolism assessment, a cortical thickness analysis, a multimodality biomarker assessment, genomic analysis, a sarcasm detection assessment, or any combination thereof. In some embodiments, the subject has an indication that warrants prophylactic treatment. In some embodiments relating to MCI, the indication is a reduction in glucose use in the brain of the subject, is a reduction in hippocampal volume in the brain of the subject, or decreased expression of synaptophysin, AMPA-type glutamate receptor subunit GluR1, GluR2, GluR3, or GluR4, an acetylcholinesterase (AChE) enzyme isoform, or at least one synapsin isoform relative to i) a previous level of expression of synaptophysin, the AMPA-type glutamate receptor subunit GluR1, GluR2, GluR3, or GluR4, the acetylcholinesterase (AChE) enzyme isoform, or the at least one synapsin isoform in the subject, or ii) the level of expression of synaptophysin, the AMPA-type glutamate receptor subunit GluR1, GluR2, GluR3, or GluR4, an acetylcholinesterase (AChE) enzyme isoform, or the at least one synapsin isoform in subjects not afflicted with MCI.

The compounds used in the methods and compositions of the present invention may be in a salt form. As used herein, a “salt” is a salt of the instant compounds which has been modified by making acid or base salts of the compounds. In the case of compounds used to treat cancer, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

### Administration

“Administering” one or more compounds used in methods of the subject invention can be effected or performed using any of the various methods and delivery systems known to those skilled in the art. The administering can be, for example, intravenous, oral, intramuscular, intravascular, intra-arterial, intracoronary, intramyocardial, intraperitoneal, and subcutaneous. Other non-limiting examples include topical administration, or coating of a device to be placed within the subject. In embodiments, administration is effected by injection or via a catheter.

The compounds used in the methods of the present invention may be administered in various forms, including those detailed herein.

The present invention provides methods of treating a disease in which a compound is administered as a monotherapy. The present invention also provides methods in which administration with a compound is supplemented with another, adjunct therapy in a subject afflicted with a disease. In some embodiments, the treatment with the compound may be administered with an adjunct therapy, i.e. the subject or subject in need of the compound is treated with or given another drug for the disease together with the compound. Treatment with the compound and the adjunct therapy can be sequential where the subject is treated first with one drug and then the other or the two drugs are given simultaneously. These can be administered independently by the same route or by two or more different routes of administration depending on the dosage forms employed.

Injectable drug delivery systems may be employed in the methods described herein include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc). Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

The dosage of the compounds administered in treatment will vary depending upon factors such as the pharmacodynamic characteristics of a specific agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the methods of the present invention may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of administration, all using dosage forms well known to those of ordinary skill in the pharmaceutical arts.

The compounds used in the methods of the present invention can be administered in a pharmaceutically acceptable carrier. As used herein, a “pharmaceutically acceptable carrier” is a pharmaceutically acceptable solvent, suspending agent or vehicle, for delivering the compounds to the subject, such as to an animal or human subject. The carrier may be liquid or solid and is selected with the planned manner of administration in mind. Liposomes are also a pharmaceutically acceptable carrier. The compounds used in the methods of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration.

The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamallar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the methods of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase subject acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, Mack Publishing Company, a standard reference text in this field.

The compounds used in the methods of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

All publications and other references mentioned herein are incorporated by reference in their entirety, as if each individual publication or reference were specifically and individually indicated to be incorporated by reference. Publications and references cited herein are not admitted to be prior art.

This invention will be better understood by reference to the Experimental Details which follow, but those skilled in the art will readily appreciate that the specific experiments detailed are only illustrative of the invention as defined in the claims which follow thereafter.

### Experimental Details

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Example 1

### Alleviation of Intracellular Aβ1-42 Accumulation

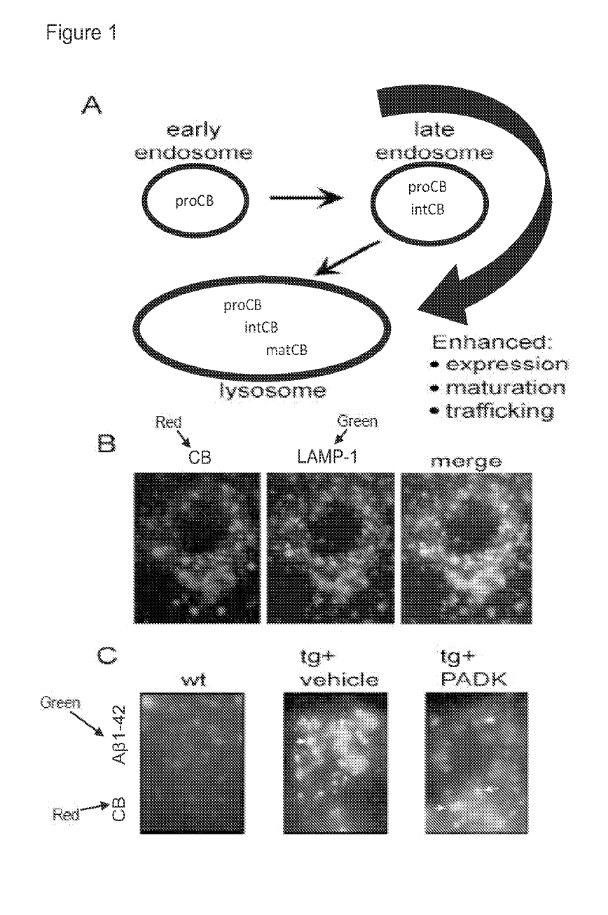

The lysosomal modulator Z-Phe-Ala-diazomethylketone (PADK) is administered to APPswe/PS1ΔE9 transgenic mice. Low-level PADK enhances cathepsin trafficking and maturation, thereby improving the clearance capacity of lysosomes (FIG. 1). It attenuates both PHF-tau and synaptic decline in the hippocampal slice model of protein accumulation (Bendiske and Bahr, 2003; Butler et al., 2005; Bahr, 2009). In hippocampal tissue of PADK-treated mice, neurons exhibited enhanced levels of cathepsin B that highly co-localized with LAMP1-positive lysosomes in neurons (FIG. 1B). In PADK-treated transgenic mice, the increase in organellar cathepsin B was associated with a decrease in intracellular Aβ1-42 in pyramidal neurons (FIG. 1C).

### Example 2

### Alleviation of Age-Associated Cognitive Impairment and Synaptic Alterations in the Fischer Rat Model of MCI

The lysosomal modulator Z-Phe-Ala-diazomethylketone (PADK) is administered to determine if age-associated synaptic alterations and cognitive impairment can be alleviated in the aged Fischer rat model by enhancing cathepsin B. In AD transgenic mouse models, the enhanced protein clearance was associated with improved synaptic integrity and cognitive ability, thus indicating the link between lysosomal capacity and the maintenance of brain function (Butler et al., 2011). In two different transgenic mouse models of AD, intracellular Aβ1-42 peptide was reduced in the hippocampus (FIG. 1C) and other brain regions, as were extracellular deposits labeled by anti-Aβ antibodies as well as H and E staining (Butler et al., 2011). Enhanced clearance by the lysosomal modulator was linked to improved synaptic integrity and cognitive ability. PADK has been the subject of intense study for proof-of-concept work in AD mouse models.

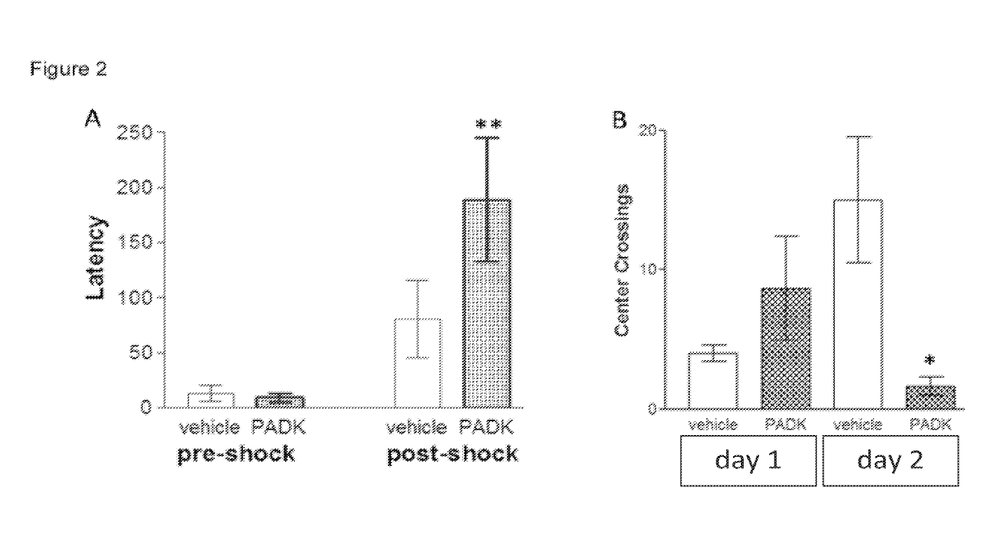

In the 11-month Fischer rats, a model of MCI and early dementia, PADK improved passive avoidance memory. The Fischer rat model exhibits a deficit in passive avoidance learning as compared to young rats, and PADK restored the learning ability to a level similar to that found in the young rats. For this study, female Fischer rats were obtained to provide lysosomal modulator testing. A passive avoidance task was used, a classic test to measure memory in rodents. The passive avoidance chamber contains a bright side and a dark side separated by a door. Rodents have a natural preference for darker environments, therefore when placed in the bright compartment they very typically enter the dark compartment quickly after the door is opened. As expected, aged rats (n=11) exhibited a deficit in shock-induced passive avoidance learning as compared to young rats (P=0.012). In a small set of aged rats compared before foot shock training, between those vehicle-treated vs. those treated daily with the lysosomal modulator PADK, no difference was evident in the short latency time before entering the opened doorway (FIG. 2A, pre-shock bars). However, the day after receiving a mild foot shock while in the dark compartment, PADK was found to improve passive avoidance behavior as compared to aged rats that received vehicle only (P=0.016; post-shock bars in FIG. 2A). These results indicate that the lysosomal modulator improves passive avoidance behavior as compared to aged rats that received vehicle only.

The animal groups were also tested across two days in the same novel field, and the number of grid crossings per 3-min period was measured to determine exploratory habituation. No significant difference was seen on day 1 of testing. On the other hand, PADK-treated rats exhibited improved habituation on the second day compared to vehicle-treated rats (FIG. 2B).

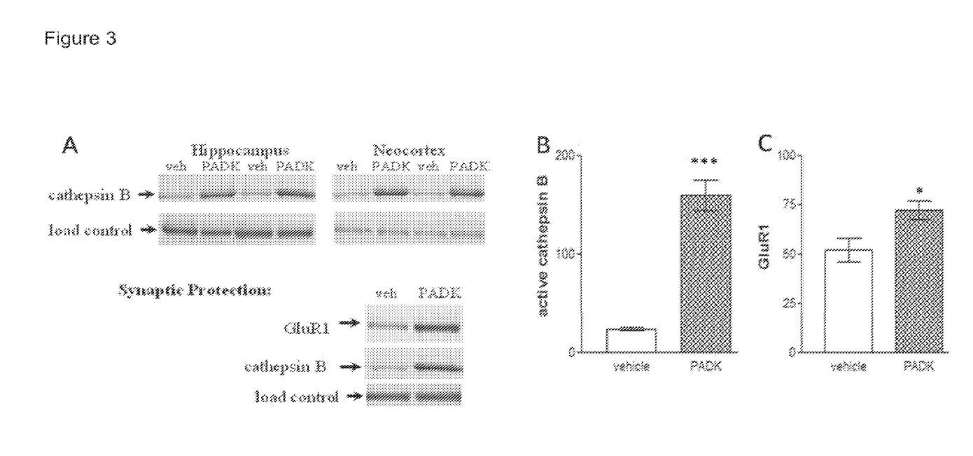

Brains were removed from the Fischer rats after the behavior studies in order to assess cathepsin B and synaptic marker levels. Corresponding with the PADK-mediated improvement in passive avoidance memory, the lysosomal modulator treatment greatly enhanced cathepsin B levels measured in brain region homogenate samples prepared after 9 days of injections (FIG. 3A upper blots, and FIG. 3B). Cathepsin B immunoreactivity was increased 4- to 8-fold in hippocampal as well as neocortical homogenates, while non-lysosomal proteins remained unchanged (gel load controls). In addition to the correspondence with improved memory, there is evidence that the lysosomal modulation corresponds with synaptic recovery, assessing the postsynaptic marker GluR1 in hippocampus (FIG. 3A lower blots, and FIG. 3C). Without wishing to be bound by any scientific theory, the results support the hypothesis that lysosomal enhancement offsets the age-related cascade of microtubule destabilization, transport failure, synaptic decline, and cognitive impairment. The data indicates a unique pharmacological strategy for the development of a disease-modifying therapy.

### Example 3

### Enhancing Cathepsin B Leads to Reductions in Multiple Types of Proteinopathy-Related Accumulation Events

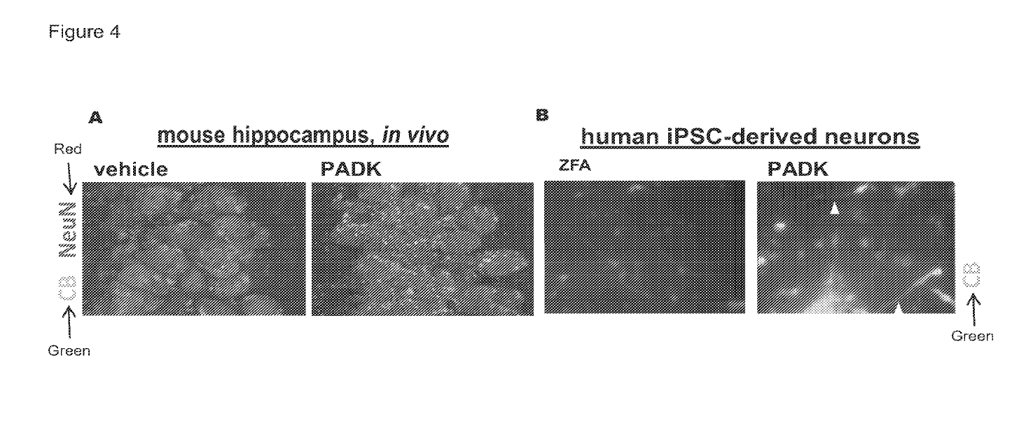

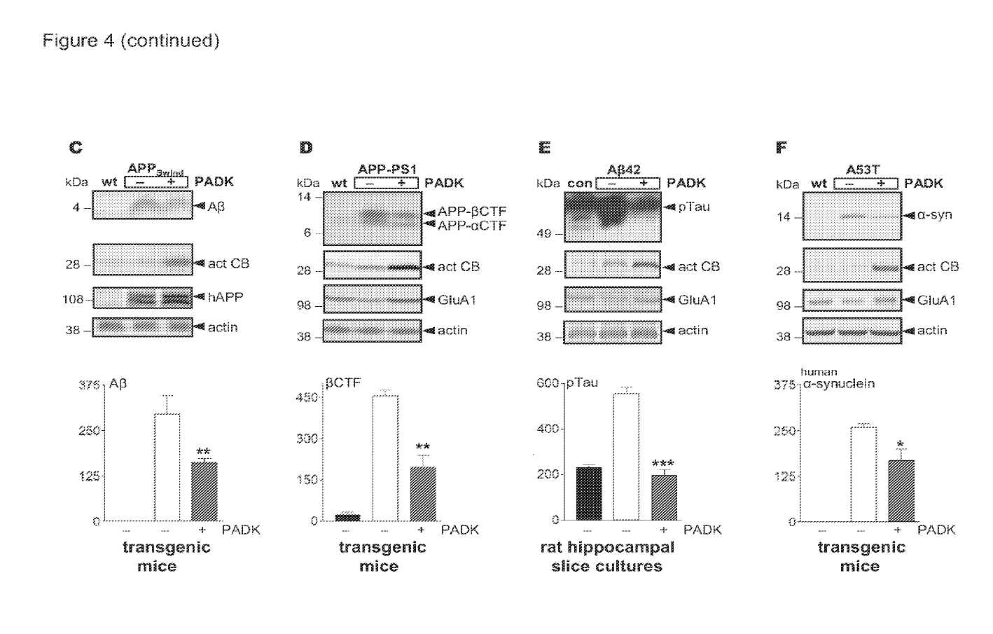

Distinct types of proteins involved in proteinopathies, namely Aβ-related peptides, tau species, and α-synuclein, appear to be targeted by a common lysosomal clearance pathway involving the enzyme cathepsin B (CatB). Previously, CatB was shown to degrade Δβ42 into smaller, less pathogenic peptides via C-terminal truncation (Mueller-Steiner et al., 2006). The study also found that genetic ablation of CatB, by crossing CatB−/− mice with hAPP transgenic mice, resulted in increased Aβ42 levels and the worsening of Aβ deposition and other AD-related pathologies. Correspondingly, Aβ deposits in AD mouse models were significantly reduced when CatB activity was enhanced by overexpressing the enzyme through lentiviral delivery (Mueller-Steiner et al., 2006) or by pharmacologically increasing the active form of CatB in neurons with PADK (FIG. 4A). The latter utilized Z-phenylalanyl-alanyl-diazomethylketone (PADK), a cathepsin modulatory compound that selectively enhances CatB levels in lysosomes, resulting in decreased intracellular Aβ, a corresponding increase in truncated Aβ38, and reduced cellular and behavioral disease parameters (Butler et al., 2006; Bahr et al., 2012). In the PADK-treated transgenic mice, reductions in Aβ peptide as well as APP carboxy-terminal fragments (αCTF and βCTF) significantly correlated with augmented levels of active CatB in the brain (FIGS. 4a and 4b). Furthermore, intracellular clearance of Aβ was associated with the reduction of extracellular Aβ deposits, indicating an equilibrium process that contributes to the dynamics of extracellular deposition (Butler et al., 2006).

The relationship between Aβ and CatB levels is similar to the relationship CatB has with the tau protein of tauopathies. Previous studies have shown that CatB inhibition and general lysosomal disruption cause an increase in both phosphorylated tau (pTau) levels and intracellular tau aggregates (Bahr, 1995). Reduction in tau aggregates and tau-related pathology also occurred when CatB activity was increased with PADK. PADK-mediated tau clearance was followed by the recovery of microtubule stability markers, transport function, and synaptic proteins (Bendiske and Bahr, 2003). In addition, the pharmacological enhancement of CatB was associated with a significant decrease in the pTau accumulation found in Aβ42-treated hippocampal slice cultures, and as in the case of the AD transgenic mouse model, the PADK effect on pTau clearance was linked to evidence of synaptic protection (FIG. 4D).

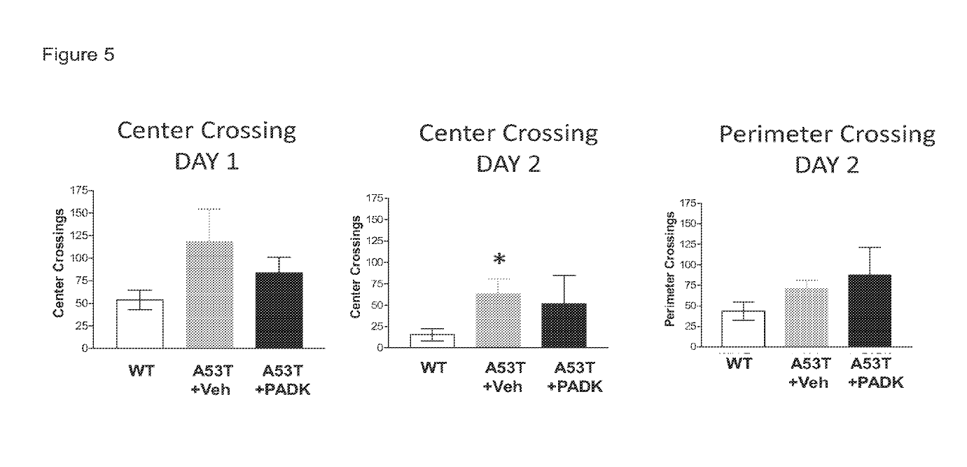

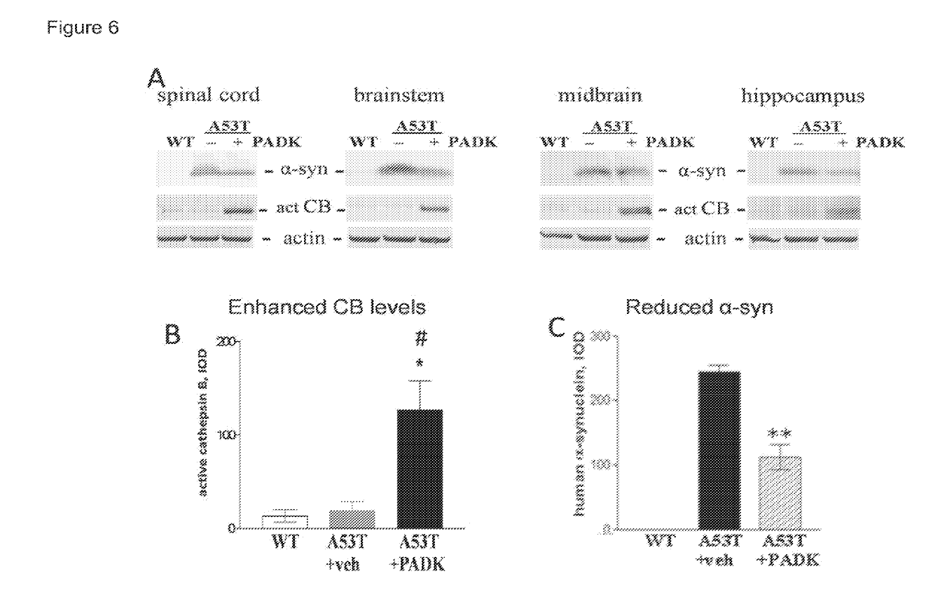

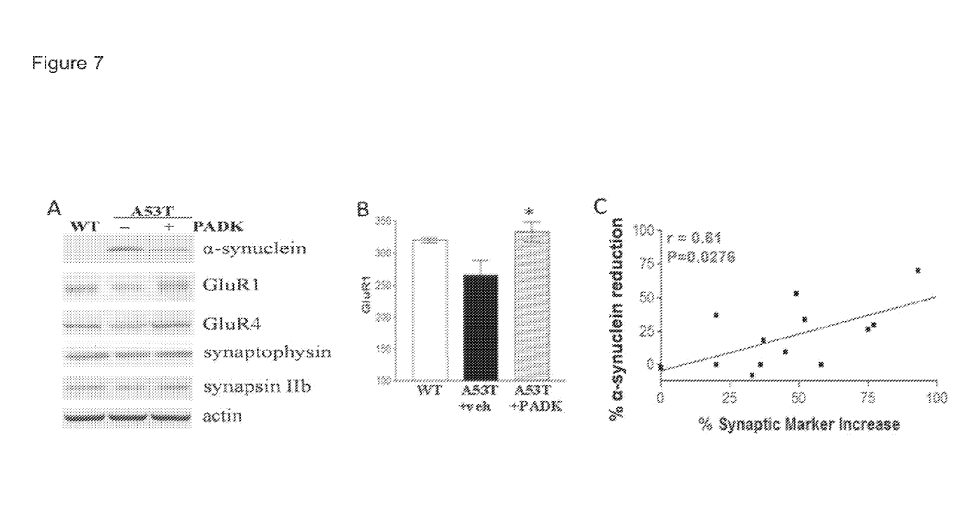

Lysosomal enhancement also promoted protein clearance and synaptic protection in the Parkinson's disease transgenic mouse model expressing mutant A53T human α-synuclein (FIG. 4E). As in the other proteinopathy models, the reduction in human α-synuclein corresponded with active CatB levels that were increased 3-6 fold in brains of the PADK-treated A53T mice. Also consistent with the other models, the degree of protein clearance significantly correlated with the extent of synaptic marker recovery. The A53T mice were used at 7-8 months of age and behavioral studies confirmed the early α-synucleinopathy was pre-symptomatic, although a subtle change in anxiety behavior was detected (FIG. 5). PADK-mediated cathepsin B modulation was evident throughout the CNS, and the enhanced levels of the enzyme's active form corresponded with improved clearance of α-synuclein (FIG. 6) and recovery of several synaptic markers (FIG. 7A). Across individual mouse samples, linear regression found that the percent recovery of synaptic marker correlates significantly with the percent reduction in human α-synuclein (FIG. 7C).

### Example 4

### Enhancing Cathepsin B in Heart Tissue for the Potential to Reduce Protein Accumulation Events Associated with Certain Cardiomyopathies

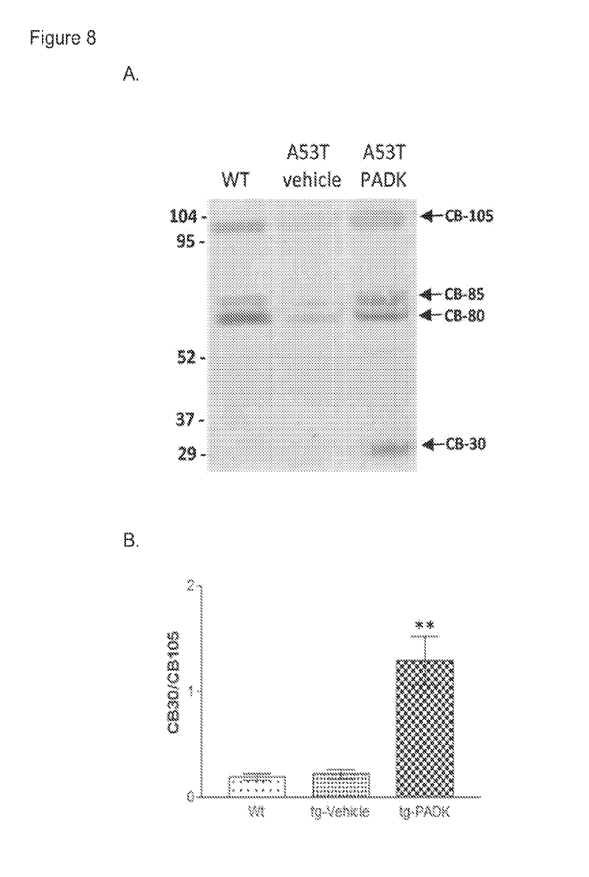

The A53T early α-synucleinopathy mouse model was injected daily with vehicle or PADK, and heart tissue was obtained to test for changes in cathepsin B. A PADK-mediated increase in the 30-kDa active form of cathepsin B was found to be robust in heart (FIG. 8). Interestingly, the A53T mice with mild protein accumulation stress exhibited declines in large cathepsin B species, and the PADK treatment returned the high molecular weight entities back to near normal levels (FIG. 8, top). Ratio measures between the 30-kDa form and the 105-kDa species indicate a unique PADK effect on cathepsin B species in the heart (FIG. 8 bar graph).

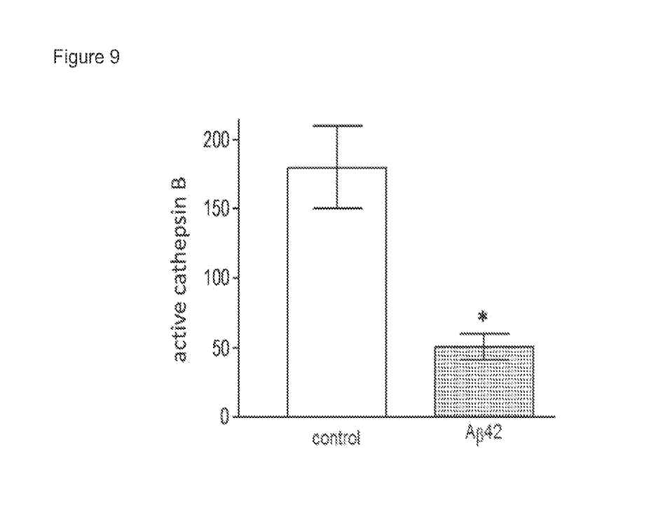

To test for changes in cathepsin B during protein accumulation induced myopathy, heart slice cultures from rat were prepared and treated with 442, the peptide that accumulates in Alzheimer's disease brains as well as in heart tissue during some forms of cardiomyopathy (Bateman et al., 2012; Sanbe et al., 2004, 2005). The Aβ42 peptide caused a significant decrease in active cathepsin B (FIG. 9). This detrimental effect on the lysosomal system suggests that a cathepsin B modulator would be beneficial to restore protein clearing capability during cardiomyopathy.

### Example 5

### The PADK Modulatory Compound Also Enhances Cathepsin B in Other Non-Brain Tissues, and it was Found not to have any Issues Across Safety Evaluation Tests

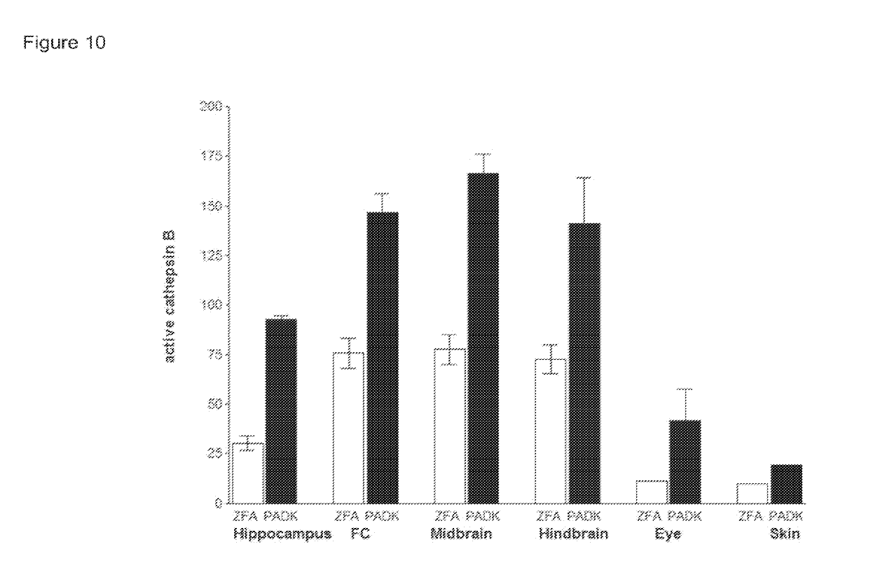

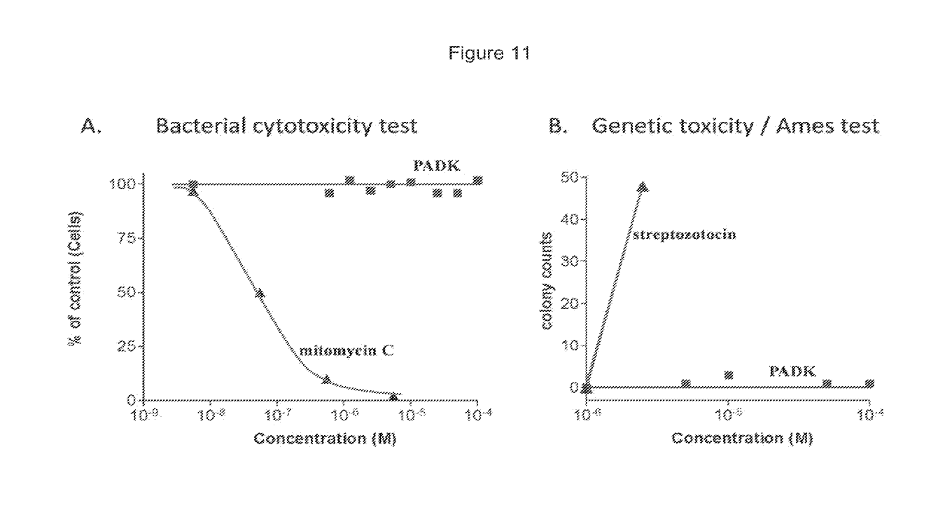

Rats were fed a peanut butter mixture containing an inactive compound (ZFA) or PADK twice daily. After 10 days, the modulatory compound PADK was found to up-regulate active cathepsin B in brain, eyes, and skin after the oral dosing (FIG. 10). Note that no evidence of adverse effects was observed during behavioral tests.

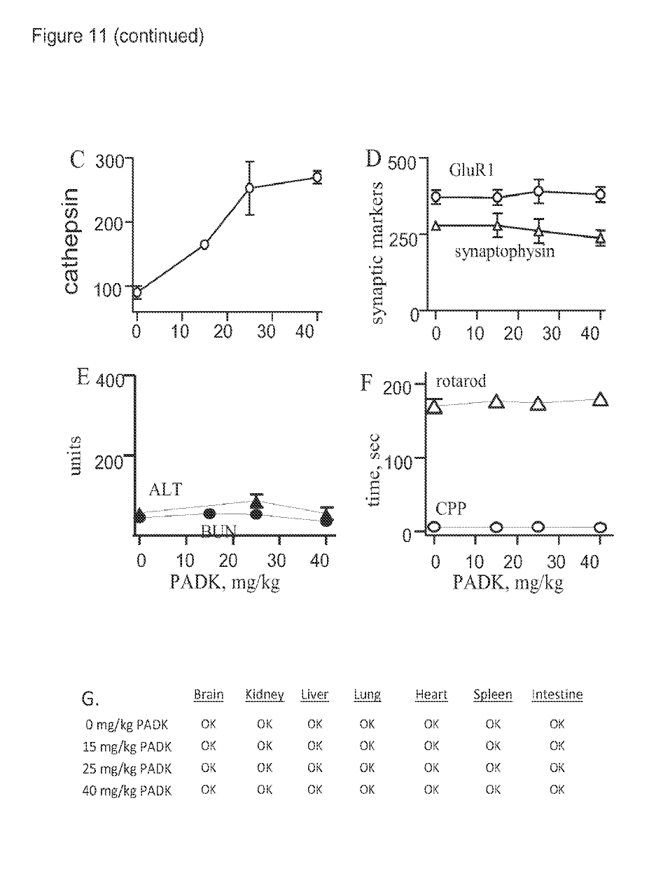

Additional safety evaluation tests with PADK were conducted. From the results, it was clear that PADK did not cause bacterial cytotoxicity (FIG. 11A) and did not produce mutant colonies in the Ames test of genetic toxicity (FIG. 11B). In PADK-injected C57BL/6 mice, a dose-dependent increase in the active cathepsin in brain samples was measured (FIG. 11C), while synaptic markers were unchanged (FIG. 11D). Blood markers were also unchanged, indicating no effect on major organs (FIG. 11E). Prior behavioral tests found no adverse effects on motor coordination or spatial memory ability (FIG. 11F). Tissue from various organs also scored as normal, thus no evidence of toxic effects (FIG. 11G).

### DISCUSSION

Lysosomes represent a major pathway for the degradation and cellular clearance of proteins. Lysosomal dysfunction has been implicated in the accumulation of various substances including Aβ peptides, paired helical filaments (PHFs), and proteoglycans. Age-related perturbation of lysosomes has been suggested to affect brain function, perhaps explaining memory decline with age. The present invention provides methods to reduce age-related accumulations to improve synaptic and cognitive integrity.

In Example 1, age-related accumulations are reduced in a test to improve synaptic and cognitive integrity in aged Fischer rats. Fischer 344 rat brains develop many of the age-related features seen in humans. Intraneuronal Aβ₁₋₄₂ is found in the brains of AD subjects and individuals with mild cognitive impairment, thus there is growing evidence that such intracellular accumulation is an early indicator of the neuronal compromise that correlates with cognitive decline. The lysosomal hydrolase cathepsin B co-localizes with intracellular Aβ1-42 and elicits cleavage into less amyloidogenic species (Mueller-Steiner et al., 2006; Butler et al., 2011). The Bahr Lab has shown that this type of Aβ detoxification can be enhanced by lysosomal modulation (Butler et al., 2011), being of particular of interest since extracellular Aβ₁₋₄₂ can be taken up by cells and sequestered in lysosomes. In fact, such uptake of monomers and small oligomers into neurons and microglia is distinctly evident, and the peptides are subsequently trafficked to lysosomes for degradation by cysteine proteases including cathepsin B (Bahr et al., 1998; Butler et al., 2011; Yang et al., 2011).

Lysosomal modulators target a novel avenue for the treatment of AD and related neurodegenerative disorders, either alone or in combination with current treatments. It is of particular interest that the first-in-class modulatory compounds promote clearance of a broad array of pathogenic proteins (as compared to strategies being developed to reduce Aβ or tau accumulations exclusively). In addition, as shown in Examples 2 and 3, the lysosomal modulator PADK was found to promote the integrity of synapses important for memory in Fischer rats and other disease models.

Positive lysosomal modulators, for instance Z-Phe-(PADK), enhance the trafficking and maturation of the lysosomal cathepsin B, thus to elicit protective clearance of toxic proteins in the brain. In aged rodents, PADK treatment improved the integrity of neuronal connections, increasing synaptic proteins well known for being reduced with aging and in association with protein accumulation pathology. Such evidence of synaptic recovery through lysosomal enhancement indicates a link between lysosomal capacity and the maintenance of brain function, providing a unique pathway to attenuate cognitive impairment and delay the age-related risk factor for dementia. Using cognitively-impaired aged Fischer rats, a new model of mild cognitive impairment (MCI) and pre-Alzheimer's disease, the studies concluded that improved memory, synaptic recovery, and dramatically up-regulated cathepsin B levels were the result of PADK treatment. The present invention provides a pharmacologically-controlled lysosomal modulation strategy against the protein accumulation stress that leads to dementia. Besides treating MCI, pre-AD, pre-Alzheimer's condition, early dementia, early AD, and related definitions, positive cathepsin B modulation is also a method to lower the risk/onset of dementia in those individuals identified as being at risk using appropriate tests (analogous to Lipitor given to individuals testing positive for high cholesterol). For treatment, brain scans, blood and/or CSF tests, and recent advances in genomics are useful for identifying individuals in a defined age bracket exhibiting early signs of protein accumulation events, neuronal connectivity compromise, synaptic decline, synaptic protein modifications, neurobiological deficits, or genetic disposition.

The aged Fischer rats provide a natural model of aging and cognitive decline and may be more translatable to humans, as compared to transgenic mouse models used previously to assess lysosomal modulators—the mouse models focus only on one particular aspect of the disease. The aged rat model for AD indeed exhibits cognitive decline. As shown herein, studies using Fischer rats from Charles River Laboratories found a deficit in a memory test among the aged rats. Moreover, administering the lysosomal modulator PADK over a 9-day period resulted in improved passive avoidance memory, recovery of a synaptic protein, and dramatically up-regulated cathepsin B levels by 4- to 8-fold (see Example 2).

Appropriate animal models are necessary in order to understand the pathogenic mechanisms of MCI and develop drugs for its treatment. The aged Fischer rat model has been indicated as an animal model of MCI, namely old age, subtle memory impairment, and mild neuropathological changes. These features occur in aging animals with normal motor activity and feeding behavior, and the memory deficits are detected by appropriately difficult behavioral tasks. Aged rats do not spontaneously develop the key Alzheimer's disease-like histopathological hallmarks, and are therefore of no use to the development of drugs targeting these pathological changes (Van Dam and De Deyn, 2011: “Ageing rodents do not spontaneously develop Alzheimer's disease-like histopathological hallmarks, and are therefore of no use to the development of drugs targeting these pathological hallmarks.”). The Fischer rat model is also unique in that reduction in nitric oxide is thought to facilitate Aβ-mediated neuropathology for an additional pathological feature of AD, however, Meyer et al. (1998) found no age-related loss of nitric oxide-producing neurons or fibers in Fischer rats. See Meyer et al. (1998): “However, we have found no age-related loss of NOS-containing HC neurons or fibers in rats.” Particularly surprising in regards to uniqueness, the Alzheimer's disease Aβ peptide acts much differently, opposite in fact, in the Fischer rat model as compared to AD mouse models (see Ruiz-Opazo et al., 2004: “We developed a transgenic Fischer rat model, TgAPPswe, that expresses the APPswe mutation driven by the platelet-derived growth factor (PDGF) promoter. To our surprise, the cognitive performance of TgAPPswe rats was significantly better than age-matched 6- and 12-mo-old control Fischer-344 rats respectively in hippocampus-dependent learning and memory tasks. In contrast, to transgenic APPswe mouse models (7), the increases in brain APP mRNA and peptide, as well as Aβ peptide levels in TgAPPswe rats, are much less and do not result in extracellular deposits”).

Concerning drug discovery, it is well accepted in the field that experimental drug treatments that improve pathological symptoms in Alzheimer's disease transgenic mouse models (models that express human proteins with mutations linked to AD) do not produce such improvements in the Fischer rat model (review by Yuede et al., 2007). For example, memantine—this is the approved drug Namenda for treating Alzheimer's disease—was found to improve spatial learning in a transgenic mouse model of Alzheimer's disease (Minkeviciene et al., 2004). However, memantine failed to significantly improve spatial memory in Fischer rats as found by one of the leading labs in the field (Barnes et al., 1996). Thus, the present invention is novel and relates to the discovery that the PADK compound that improves Alzheimer's disease symptoms in transgenic mouse models (see Butler et al., 2011) also improves memory and synaptic marker levels in aged Fischer rats (FIGS. 2 and 3). Concluded from the unexpected findings is that the PADK compound, and related cathepsin B enhancing compounds, acts in Fischer rats in more and/or different ways in which it acts in Alzheimer's disease transgenic mouse models.

The cognitively-impaired aged rats also show early pathological features common to human AD, including brain atrophy, synaptic abnormalities, and changes in synaptic plasticity. Aging is the single most important contributing factor to the development of AD. Specific synaptic proteins have been identified that decrease with brain aging, including synaptophysin (Masliah et al., 1993; Callahan et al., 1999; Coleman et al., 2004), AMPA-type glutamate receptor subunit GluR1 (Bahr et al., 1992), acetylcholinesterase (AChE) enzyme isoforms (Skau and Triplett, 1998), and NCAM (Bahr et al. 1993), the neural cell adhesion molecule that has been implicated in neurotransmission regulation events (Vaithianathan et al., 2004) found to be linked to memory encoding (Suppiramaniam et al., 2001). Many of the listed synaptic proteins and other signs of synaptic integrity are reduced in AD, mild cognitive impairment, and/or during protein accumulation pathology (Sze et al., 1997; Bahr et al., 1998; Wakabayashi et al., 1999; Ginsberg et al., 2000; Bahr and Bendiske, 2002; Bendiske et al., 2002; Takahashi et al., 2002; Coleman et al., 2004; Kanju et al., 2007; Scheff et al., 2007).

Lysosomes provide broad degradation pathways, and the catabolic processes involve cooperation between autophagic trafficking and enzyme delivery for efficient degradation and turnover of proteins and other material. Altered protein processing capability in lysosomes has been suggested to affect brain function during normal aging as well as in age-related diseases. Lysosomal instability is a distinct feature of brain aging (Brunk and Brunk, 1972; Kikugawa et al., 1994), resulting in gradual changes and increased risk for protein accumulations and aggregated protein stress responses. As disclosed herein, a pharmacologically-controlled lysosomal modulation strategy was developed against the protein accumulation stress and gradual synaptic pathology of the aged brain. Enhancement of the lysosomal system has been suggested as a plausible strategy to reduce aberrant protein accumulation in age-related neurodegenerative disorders (Bahr, 2003; Bendiske and Bahr, 2003; Lee et al., 2004; Mueller-Steiner et al., 2006; Bahr et al., 2009). Butler et al. (2011) demonstrated that the pharmacologically-controlled modulation of the lysosomal system with PADK promotes clearance of Aβ species in two mouse models of AD. The findings also indicate that reducing intracellular protein accumulation stress protects against AD-type synaptic changes. Reduction in extracellular deposits occurred in correspondence with the reduced intraneuronal Aβ, providing evidence of an equilibrium relationship between secreted Aβ peptides and the extracellularly deposited species. The lysosomal modulator PADK markedly up-regulated the active form of cathepsin B. The improved synaptic integrity and associated cognitive improvement were likely due to the reduction in protein accumulation events linked to the pathogenic sequelae underlying transport deficits, axonopathy, and synaptic compromise.

Heart failure is the leading cause of death in the developed world, and it represents a common endpoint for several diseases, including hypertension, coronary artery disease, and the cardiomyopathies. Amyloid oligomers, similar to the toxic entities found in Alzheimer's disease subjects, are present in cardiomyocytes derived from human heart-failure subjects and in animal models of cardiomyopathy. The increased expression of some lysosomal proteins may contribute to heart failure or, alternatively, may represent a compensatory feedback mechanism to enhance protein clearance in response to pathogenic protein accumulation.

Blocking cardiac amyloid oligomer formation, even after cardiac dysfunction presents, may be a therapeutic strategy for the types of cardiac disease in which significant amyloid accumulation occurs (Sanbe et al., 2004, 2005). The data of Sanbe et al, point to a pathogenic process for cardiovascular disease and provide a link to the degenerative amyloidoses. The lysosomal enzyme cathepsin B degrades Aβ42 by C-terminal truncation, lowering levels in mice expressing hAPP (Mueller-Steiner et al., 2006; Butler et al., 2011; Wang et al., 2012) and reducing both plaque load in the brain and behavioral and synaptic marker deficits. However, myocardial expression of cathepsin B has been found in subjects with heart failure, which has been suggested to relate to the genesis and development of heart failure (Ge et al. (2006) “Enhanced myocardial cathepsin B expression in subjects with dilated cardiomyopathy” Eur J Heart Fail. 8(3):284-9). Up-regulation of the intermediate filament protein desmin occurs in cardiac disorders such as cardiac hypertrophy and congestive heart failure (CHF) (Heling et al 2000), and desmin mutations are associated with desmin-related cardiomyopathy (DRM) and idiopathic dilated cardiomyopathy (Li et al 1999, Sojberg et al 1999). Mutations in other proteins also have been associated with the DRMs, and genetic evidence has linking an R120G mutation in α-B-crystallin (CryAB, CryAB^{R120G}) to human DRM (Vicart et al 1998). Cardiac-restricted transgenic (TG) expression of CryAB^{R120G} was sufficient to cause heart failure in a mouse model (Want et al 2001).

The present invention provides methods for reducing protein accumulation and aggregation even after cardiac dysfunction presents. In some embodiments, the level of cardiac amyloid oligomer formation, and clearing of cardiac amyloid oligomers, even after cardiac dysfunction presents, as a therapeutic strategy in DRM as well as in other types of cardiac disease in which significant amyloid accumulation occurs. As disclosed herein, enhancing cathepsin B levels with a compound like PADK is useful for treating amyloid-related cardiomyopathies. This is surprising since cathepsin B has been suggested to contribute to heart failure. Surprisingly, the proteomic makeup of cathepsin B species in heart tissue is unique compared to brain and other tissues. As disclosed herein for the first time, PADK enhances the ratio between active cathepsin B form of 30 kDa and the precursor of >100 kDa (p<0.01). This is unique compared to other tissues.

Previous work demonstrated that cells are capable of clearing preformed α-synuclein aggregates via the lysosomal degradation pathway (Lee et al., 2004), suggesting that enhancing lysosomal function may be a potential therapeutic strategy to halt or prevent the pathogenesis of Parkinson's disease and other α-synucleinopathy diseases. The Bahr-Ikonne invention, for the first time, shows that a compound that enhances the cellular level of active cathepsin B improves human α-synuclein clearance and synaptic integrity in a transgenic mouse model prior to developing the behavioral symptoms of Parkinson's disease (FIGS. 6 and 7). In addition, it is unexpected that a compound that promotes Aβ42 clearance also promotes α-synuclein clearance given the distinct properties of Aβ42 and α-synuclein (see Suh and Checler, 2002).

Multiple proteins implicated in neurodegenerative diseases accumulate in axons after brain trauma in humans (Uryu et al. 2007). Studies in animal models have shown that traumatic brain injury (TBI) induces the rapid accumulation of many of the same key proteins that form pathologic aggregates in neurodegenerative diseases. Here, we examined whether this rapid process also occurs in humans after TBI. Brain tissue from 18 cases who died after TBI and from 6 control cases was examined using immunohistochemistry. Following TBI, widespread axonal injury was persistently identified by the accumulation of neurofilament protein and amyloid precursor protein (APP) in axonal bulbs and varicosities. Axonal APP was found to co-accumulate with its cleavage enzymes, beta-site APP cleaving enzyme (BACE), presenilin-1 (PS1) and their product, amyloid-beta (Abeta). In addition, extensive accumulation of alpha-synuclein (alpha-syn) was found in swollen axons and tau protein was found to accumulate in both axons and neuronal cell bodies. These data show rapid axonal accumulation of proteins implicated in diseases including Alzheimer's disease and the α-synucleinopathies. The cause of axonal pathology can be attributed to disruption of axons due to trauma, or as a secondary effect of raised intracranial pressure or hypoxia. Such axonal pathology in humans may provide a unique environment whereby co-accumulation of APP, BACE, and PS1 leads to intra-axonal production of Abeta as well as accumulation of α-synuclein and tau. This process may have important implications for survivors of TBI who have been shown to be at greater risk of developing neurodegenerative diseases.

Cutaneous exposure to chronic solar WA-radiation is a causative factor in dermal photodamage and photoaging. Previous studies support the hypothesis herein that cathepsin B is a crucial target of UVA-induced photo-oxidative stress causatively involved in dermal photodamage through the impairment of lysosomal removal of lipofuscin, with cathepsin B inactivation as a novel molecular mechanism involved in the WA-induced skin photodamage (Lamore et al., 2010; Lamore and Wondrak, 2012). In addition, a study of scratch wounding of keratinocytes demonstrated that cathepsin B is essential during initial stages of wound healing (Büth et al., 2007).

### REFERENCE

1. Bahr B A, Park G Y, Singh M, and Lynch G (1994) Induction of Aging and Excitotoxicity Processes in Long-Term Hippocampal Slice Cultures, Mol. Biol. Cell 5 (Suppl.), 468a.
2. Bahr B A (1995) Long-term hippocampal slices: A model system for investigating synaptic mechanisms and pathologic processes. J Neurosci Res 42:294-305.
3. Bahr B A, Wisniewski M L, and Butler D (2012) Positive lysosomal modulation as a unique strategy to treat age-related protein accumulation diseases. Rejuvenation Res 15:189-197.
4. Barnes, C. A. Danysz, W., & Parsons, C. G. (1996). Effects of the Uncompetitive NMDA Receptor Antagonist Memantine on Hippocampal Long-term Potentiation, Short-term Exploratory Modulation and Spatial Memory in Awake, Freely Moving Rats. European Journal of Neuroscience, 8(3), 565-571. Note: Memantine failed to significantly improve maze performance in Fischer rats.
5. Bateman R J, Xiong C, Benzinger T L, Fagan A M, Goate A, Fox N C, Marcus D S, Cairns N J, Xie X, Blazey T M, Holtzman D M, Santacruz A, Buckles V. Oliver A, Moulder K, Aisen P S, Ghetti B, Klunk W E, McDade E, Martins R N, Masters C L, Mayeux R, Ringman J M, Rossor M N, Schofield P R, Sperling R A, Salloway S, Morris J C; and the Dominantly Inherited Alzheimer Network (2012) Clinical and biomarker changes in dominantly inherited Alzheimer's disease. New England Journal of Medicine 367:795-804. doi: 10.1056/NEJMoa1202753.
6. Bendiske J, Bahr B A (2003) Lysosomal activation is a compensatory response against protein accumulation and synaptopathogenesis—An approach for slowing Alzheimer's disease? J Neuropathol Exp Neurol 62:451-463.
7. Bern, A. W., Bauer, A. Q., Stewart, F. R., White, B. R., Cirrito, J. R., Raichle, M. E., Culver, J. P., and Holtzman, D. M. (2012). Bidirectional Relationship between Functional Connectivity and Amyloid-8 Deposition in Mouse Brain. The Journal of Neuroscience. 32(13): 4334-4340; doi: 10.1523/JNEUROSCI.5845-11.2012
8. Bolmont T, Bouwens A, Pache C, Dimitrov M, Berclaz C, Villiger M, Wegenast-Braun B M, Lasser T, and Fraering P C (2012). Label-Free Imaging of Cerebral (beta)-Amyloidosis with Extended-Focus Optical Coherence Microscopy. Neurosci. 32: 14548-14556.
9. Büth H, Luigi Buttigieg P, Ostafe R, Rehders M, Dannenmann S R, Schaschke N, Stark H J, Boukamp P, Brix K (2007) Cathepsin B is essential for regeneration of scratch-wounded normal human epidermal keratinocytes. Eur J Cell Biol 86:747-761.
10. Butler D, Hwang J, Estick C, Nishiyama A, Kumar S S, Baveghems C, Young-Oxendine H B, Wisniewski M L, Charalambides A, and Bahr B A (2011) Protective effects of positive lysosomal modulation in Alzheimer's disease transgenic mouse models. PLoS One 6: e20501.
11. Dentchev T, Milam A H, Lee V M, Trojanowski J Q, Dunaief J L (2003) Amyloid-beta is found in drusen from some age-related macular degeneration retinas, but not in drusen from normal retinas. Mol Vis, 9:184-190.
12. Guan X. DIuzen D E. Age-related changes of social memory/recognition in male Fischer 344 rat.Behav Brain Res. 1994:61:87-90.
13. Hahn C, Lim H-K, Won W Y, Ahn K J, Jung W-S, et al. (2013) Apathy and White Matter Integrity in Alzheimer's Disease: A Whole Brain Analysis with Tract-Based Spatial Statistics, PLoS ONE 8(1): e53493. doi:10.1371/journal.pone.0053493
14. Hautala K (2010) UKNOW University of Kentucky News. In Alzheimer's imaging study identifies brain changes. http://uknow.uky.edu/content/alzheimers-imaging-study-identifies-brain-changes. Available from: uknow.uky.edu/content/alzheimers-imaging-study-identifies-brain-changes.
15. Heling, A., Zimmermann, R., Kostin, S., Maeno, Y., Hein, S., Devaux, B., Bauer, E., Klovekorn, W. P., Schlepper, M., Schaper, W. & Schaper, J. (2000) Increased expression of cytoskeletal, linkage, and extracellular proteins in failing human myocardium. Circ. Res. 86, 846-853.
16. Isas J M, Luibl V, Johnson L V, Kayed R, Wetzel R, Glabe C G, Langen R, Chen J (2010) Soluble and mature amyloid fibrils in drusen deposits. Invest Ophthalmol Vis Sci, 51(3):1304-1310.
17. Johnson L V, Leitner W P, Rivest A J, Staples M K, Radeke M J, Anderson D H (2002) The Alzheimer's Abets-peptide is deposited at sites of complement activation in pathologic deposits associated with aging and age-related macular degeneration. Proc Natl. Acad Sci USA, 99(18):11830-11835.
18. Kaarniranta K, Salminen A, Haapasalo A, Soininen H, Hiltunen M (2011) Age-Related Macular Degeneration (AMD): Alzheimer's Disease in the Eye? J Alzheimers Dis, 24(4):615-631.
19. Lamore S D and Wondrak G T (2012) Autophagic-lysosomal dysregulation downstream of cathepsin B inactivation in human skin fibroblasts exposed to OVA. Photochem Photobiol Sci 11:163-172.
20. Lamore S D, Qiao S, Horn D, and Wondrak G T (2010) Proteomic identification of cathepsin B and nucleophosmin as novel UVA-targets in human skin fibroblasts. Photochem Photobiol 86:1307-1317.
21. Lee H J, Khoshaghideh F, Patel S. Lee S J (2004) Clearance of α-synuclein oligomeric intermediates via the lysosomal degradation pathway. J Neurosci 24: 1888-1896.
22. Li, D., Tapscoft, T., Gonzalez, O., Burch, P. E., Quinones, M. A., Zoghbi, W. A., Hill, R., Bachinski, L. L., Mann, D. L. & Roberts, R. (1999) Desmin mutation responsible for idiopathic dilated cardiomyopathy. Circulation 100, 461-464.
23. Luibl V, Isas J M, Kayed R, Glabe C G, Langen R, Chen J: Drusen deposits associated with aging and age-related macular degeneration contain nonfibrillar amyloid oligomers. J Clin Invest 2006, 116(2):378-385.
24. Mariani E, Monastero R, Mecocci P. Mild cognitive impairment: a systematic review. J Alzheimers Dis. 2007 August; 12(1):23-35
25. Meyer R C, Spangler E L, Kametani H, Ingram D K (1998) Age-associated memory impairment. Assessing the role of nitric oxide. Ann N Y Aced Sci. 854:307-17
26. Minkeviciene, R., Banerjee, P., Tanila, H., 2004. Memantine improves spatial learning in a transgenic mouse model of Alzheimer's disease. J. Pharmacol. Exp. Ther. 311, 677-682.\
27. Mueller-Steiner S, Zhou Y, Roberson E D, Sun B, Chen J, Wang X, Yu G, Esposito L, Mucke L, Gan L (2006) Antiamyloidogenic and neuroprotective functions of cathepsin B: Implications for Alzheimer's disease. Neuron 51:703-714.
28. Petersen R C, Roberts R O, Knopman D S, Boeve B F, Geda Y E, Ivnik R J, Smith G E, Jack C R Jr. Mild cognitive impairment: ten years later. Arch Neural. 2009 December; 66(12):1447-55.
29. Raymond, Joan. “Sarcastic or Serious? Missing Subtle Cues May Signal Dementia.” Msnbc.com. NBCNews, 18 Apr. 2011. Web. 6 Apr. 2013
30. Ruiz-Opazo N, Kosik K S, Lopez L V, Bagamasbad P, Ponce L R, Herrera V L (2004) Attenuated hippocampus-dependent learning and memory decline in transgenic TgAPPswe Fischer-344 rats. Mol Med. 10:36-44.
31. Sanbe A, Osinska H, Saffitz J E, Glabe C G, Kayed R, Maloyan A, Robbins J (2004) Desmin-related cardiomyopathy in transgenic mice: a cardiac amyloidosis. Proc Natl Acad Sci U.S.A. 101:10132-6.
32. Sanbe A, Osinska H, Villa C, Gulick J, Klevitsky P. Glade C G, Kayed R, and Robbins J (2005) Reversal of amyloid-induced heart disease in desmin-related cardiomyopathy. Proc Natl Acad Sci U.S.A. 102:13592-7.
33. Scheff S W, Price D A, Schmitt F A, DeKosky S T, Mufson E J. Synaptic alterations in CA1 in mild Alzheimer disease and mild cognitive impairment. Neurology. 2007 May 1; 68(18):1501-8.
34. Skau K A, Triplett C G. Age-related changes in activity of Fischer 344 rat brain acetylcholinesterase molecular forms. Mol Chem Neuropathol. 1998 August-December; 35(1-3):13-21.
35. Sjoberg, G., Saavedra-Matiz, C. A., Rosen, D. R., Wijsman, E. M., Borg, K., Horowitz, S. H. & Sejersen, T. (1999) A missense mutation in the desmin rod domain is associated with autosomal dominant distal myopathy, and exerts a dominant negative effect on filament formation. Hum. Mol. Genet. 8, 2191-2198.
36. Sub Y H, Checler F. Amyloid precursor protein, presenilins, and alpha-synuclein: molecular pathogenesis and pharmacological applications in Alzheimer's disease. Pharmacol Rev. 2002 September; 54(3):469-525.
37. Takefumi K., Toshiyuki T., Tsugio K. Age-related working memory deficits in the allocentric place discrimination task. Neurobiol Aging. 1999; 20:629-636.
38. Uryu et al, “Multiple proteins implicated in neurodegenerative diseases accumulate in axons after brain trauma in humans” Exp Neurol. 2007 December; 208(2):185-92. Epub 2007 Jul. 10,
39. Van Dam D and De Deyn P P (2011) Animal models in the drug discovery pipeline for Alzheimer's disease. British Journal of Pharmacology 164:1285-1300.
40. Vicart, P., Caron, A., Guicheney, P., Li, Z., Prevost, M. C., Faure, A., Chateau, D., Chapon, F., Tome, F., Dupret, J. M., et al. (1998) A missense mutation in the alphaB-crystallin chaperone gene causes a desmin-related myopathy. Nat. Genet. 20, 92-95.
41. Viswanathan K, Hoover D J, Hwang J, Wisniewski M L, Ikonne U S, Bahr B A, and Wright D L (2012) Nonpeptidic lysosomal modulators derived from Z-Phe-Ala-diazomethylketone for treating protein accumulation diseases. ACS Med Chem Lett 3:920-924.
42. Wang C, Sun B, Zhou Y, Grubb A, Gan L (2012) Cathepsin B degrades amyloid-3 in mice expressing wild-type human amyloid precursor protein. J Biol Chem. 287 (47): 39834-41.
43. Wang, X., Osinska, H., Klevitsky, R., Gerdes, A. M., Nieman, M., Lorenz, J., Hewett, T. & Robbins, J. (2001) Expression of R120G-alphaB-crystallin causes aberrant desmin and alphaB-crystallin aggregation and cardiomyopathy in mice. Circ. Res, 89, 84-91.
44. Yuede, C. M., Dong, H., & Csernansky, J. G. (2007). Anti-dementia drugs and hippocampal-dependent memory in rodents. Behavioural pharmacology, 18(5-6), 347-362. The Yuede review covers many studies that indicate several drug treatments that work in AD mice do not work in Fischer rat model.
45. Zhizhang Dong, Juan Li, Yunxia Leng, Xuerong Sun, Hulling Hu, Yuan He, Zhiqun Tan, and Jian Ge (2012) Cyclic intensive light exposure induces retinal lesions similar to age-related macular degeneration in APPswe/PS1 bigenic mice. BMC Neurosci.; 13: 34.
46. Press Release, National Institute of Neurological Disorders and Stroke, National Institutes of Health, “NIH-funded researchers create next-generation Alzheimer's disease model” For release: Apr. 9, 2013, available at www.ninds.nih.gov/news_and_events/news_articles/rat_AD_model.ht m; “We focused on Fischer 344 rats because their brains develop many of the age-related features seen in humans,” said Dr. Town, who conducted the study while working as a professor of Biomedical Sciences at Cedars-Sinai Medical Center and David Geffen School of Medicine at the University of California, Los Angeles.

1-146. (canceled)

147. A method for treating a subject afflicted with a protein accumulation disease, Mild Cognitive Impairment (MCI), or Traumatic Brain Injury (TBI), comprising administering to the subject at least one compound that increases the level of active cathepsin B in cells of the subject, or a pharmaceutically acceptable salt or ester thereof, in an amount that is effective to treat the subject.

148. The method of claim 147, wherein the subject is afflicted with a protein accumulation disease.

149. The method of claim 148, wherein the protein accumulation disease is
i) brain protein accumulation disease, and the cells are brain cells;
ii) cardiomyopathy, and the cells are heart cells;
iii) macular degeneration, and the cells are eye cells; or
iv) skin damage, and the cells are skin cells or hypodermis cells.

150. The method of claim 149, wherein the protein accumulation disease is a brain protein accumulation disease, and wherein the brain protein accumulation disease is Alzheimer's disease, pre-Alzheimer's disease, early Alzheimer's disease, early-onset Alzheimer's disease, late-onset Alzheimer's disease, Huntington's disease, α-synucleinopathy, dementia, or amyotrophic lateral sclerosis.

151. The method of claim 150, wherein the brain protein accumulation disease is α-synucleinopathy, and wherein the α-synucleinopathy is Parkinson's Disease.

152. The method of claim 147, wherein the at least one compound is orally administered to the subject in a pharmaceutically acceptable carrier.

153. The method of claim 147, wherein the at least one compound is Z-phenylalanyl-alanyl-diazomethylketone (PADK) or a PADK analogue, or a pharmaceutically acceptable salt or ester thereof.

154. The method of claim 153, wherein the at least one compound is a PADK analogue.

155. The method of claim 154, wherein the PADK analogue is Z-L-phenylalanyl-D-alanyl-diazomethylketone (PdADK) Z-D-phenylalanyl-L-alanyl-diazomethylketone (dPADK) or Z-D-phenylalanyl-D-alanyl-diazomethylketone (dPdADK).

156. The method of claim 154, wherein the PADK analogue is Z-phenylalanyl-phenylalanyl-diazomethylketone (PPDK), or a pharmaceutically acceptable salt or ester thereof.

157. The method of claim 154, wherein the PADK analogue is Z-L-phenylalanyl-D-phenylalanyl-diazomethylketone (PdPDK) Z-D-phenylalanyl-L-phenylalanyl-diazomethylketone (dPPDK) or Z-D-phenylalanyl-D-phenylalanyl-diazomethylketone (dPdPDK).

158. The method of claim 154, wherein the PADK analogue has the structure: 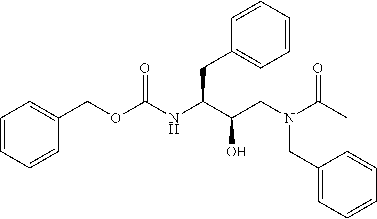 [SMILES: CC(=O)N(Cc1ccccc1)C[C@@H](O)[C@H](Cc1ccccc1)NC(=O)OCc1ccccc1]
or a pharmaceutically acceptable salt or ester thereof.

159. The method of claim 154, wherein the PADK analogue has the structure: 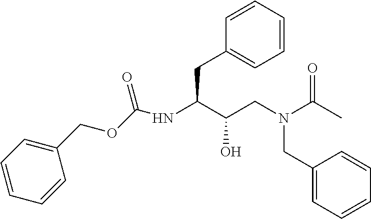 [SMILES: CC(=O)N(Cc1ccccc1)C[C@H](O)[C@H](Cc1ccccc1)NC(=O)OCc1ccccc1]
or a pharmaceutically acceptable salt or ester thereof.

160. The method of claim 154, wherein the PADK analogue has the structure: 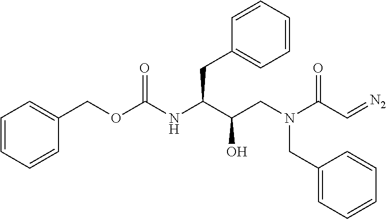
or a pharmaceutically acceptable salt or ester thereof.

161. The method of claim 147, wherein the at least one compound is a lysosomal modulator having the structure: 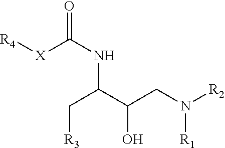
wherein R₁, R₂, R₃, and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring; and
wherein substituted means having one or more atoms replaced with one or more identical or different substituents selected from the group consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkylthio, alkenylthio, alkynylthio, sulfonyl, alkyl sulfonyl, sulfinyl, and alkylsulfinyl,
wherein X is a heteroatom including but not limited to O, N, or S,
or a pharmaceutically acceptable salt or ester thereof.

162. The method of claim 147, wherein the at least one compound is leupeptin, deacetyl-leupeptin, E-64, E-64c, E-64d, diazoacetyl-DL-2-aminohexanoic acid methyl ester, bafilomycin A1, glycyl-phenylalanyl-glycine-aldehyde semicarbazone, or pepstatin A, or a pharmaceutically acceptable salt or ester thereof.

163. The method of claim 147, wherein the at least one compound is a combination of two, three, four, or more of:
i) PADK or a PADK analogue, or a pharmaceutically acceptable salt or ester thereof;
ii) a lysosomal modulator having the structure: 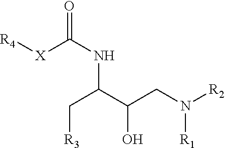
wherein R₁ and R₂ independently are halogen; hydroxyl; alkoxy; acyl; cyano; nitro; amino; substituted or unsubstituted alkyl, alkenyl, or alkynyl groups; or substituted or unsubstituted aromatic or cyclic aliphatic groups, which may include one or more heteroatoms in the ring;
wherein, R₃ and R₄ independently are hydrogen; halogen; hydroxyl; alkoxy; acyl; cyano; nitro; amino; substituted or unsubstituted alkyl, alkenyl, or alkynyl groups; or substituted or unsubstituted aromatic or cyclic aliphatic groups which may include one or more heteroatoms in the ring;
wherein substituted means substituted with one or more identical or different substituents selected from the group consisting of halogen, hydroxyl, alkyl, alkenyl, alkynyl, alkoxy, cycloalkoxy, haloalkyl, haloalkoxy, acyl, amino, alkylamino, dialkylamino, nitro, cyano, thio, alkyl thio, alkenylthio, alkynylthio, sulfonyl, alkylsulfonyl, sulfinyl, and alkylsulfinyl; and
wherein X is a heteroatom selected from O, N, or S, or a pharmaceutically acceptable salt or ester thereof;
iii) PPDK or a pharmaceutically acceptable salt or ester thereof;
iv) a PADK analogue having the structure: 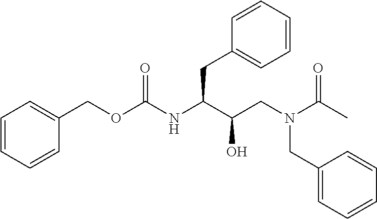 [SMILES: CC(=O)N(Cc1ccccc1)C[C@@H](O)[C@H](Cc1ccccc1)NC(=O)OCc1ccccc1]
or a pharmaceutically acceptable salt or ester thereof;
v) a PADK analogue having the structure: 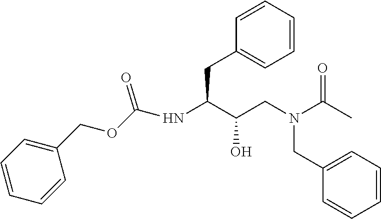 [SMILES: CC(=O)N(Cc1ccccc1)C[C@H](O)[C@H](Cc1ccccc1)NC(=O)OCc1ccccc1]
or a pharmaceutically acceptable salt or ester thereof;
vi) a PADK analogue having the structure: 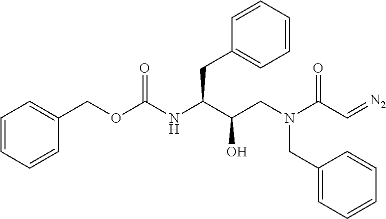
or a pharmaceutically acceptable salt or ester thereof;
vii) leupeptin or a pharmaceutically acceptable salt or ester thereof;
viii) deacetyl-leupeptin or a pharmaceutically acceptable salt or ester thereof;
ix) E-64 or a pharmaceutically acceptable salt or ester thereof;
x) E-64c or a pharmaceutically acceptable salt or ester thereof;
xi) E-64d or a pharmaceutically acceptable salt or ester thereof;
xii) diazoacetyl-DL-2-aminohexanoic acid methyl ester or a pharmaceutically acceptable salt or ester thereof;
xiii) bafilomycin A1 or a pharmaceutically acceptable salt or ester thereof;
xiv) glycyl-phenylalanyl-glycine-aldehyde semicarbazone or a pharmaceutically acceptable salt or ester thereof; or
xv) pepstatin A or a pharmaceutically acceptable salt or ester thereof,
each in an amount that when administered together is effective to treat the subject, or increase the level of active cathepsin B in brain, heart, eye, or skin cells of the subject.

164. The method of claim 147, wherein the at least one compound increases the level of active cathepsin B in brain, heart, eye, or skin cells at least about 1.5, 2, 3, 4, 5, 6, 7, 8, 9, 10, 11, 12, 14, 14, or 15-fold.

165. The method of claim 147, wherein the at least one compound increases cathepsin B trafficking and/or maturation in brain, heart, eye, or skin cells.

166. A composition for treating a subject afflicted with a protein accumulation disease, Mild Cognitive Impairment (MCI), or Traumatic Brain Injury (TBI), comprising the at least one compound from the method of claim 147 and a pharmaceutically acceptable carrier.
